(19) United States
(12) Patent Application Publication
(10) Pub. No.: US 2009/0318342 A1
(43) Pub. Date: Dec. 24, 2009

(54) Compounds

(75) Inventors: Hideaki Nagase, London (GB); Keith Brew, Boca Raton, FL (US)

Correspondence Address: NIKOLAI & MERSEREAU, P.A., 900 SECOND AVENUE SOUTH, SUITE 820, MINNEAPOLIS, MN, 55402, US

(73) Assignee: IMPERIAL INNOVATIONS LIMITED, London (GB)

(21) Appl. No.: 11/989,673

(22) PCT Filed: Jul. 28, 2006
(86) PCT No.: PCT/US2006/029726

§ 371 Date: Mar. 10, 2009

Related U.S. Application Data
(60) Provisional application No. 60/704,015, filed on Jul. 29, 2005.

(51) Int. Cl. A61K 38/17; C07K 14/00; C07H 21/04; C12N 5/10; C12P 21/02; C12Q 1/37; A61P 29/00; A61P 31/00; A61P 37/00; A61P 27/02; A61P 19/02

(52) U.S. Cl. 514/12; 530/350; 536/23.5; 435/325; 435/69.1; 435/24

(57) ABSTRACT

A mutant TIMP-3 (Tissue Inhibitor of MetalloProteinase-3) polypeptide wherein an additional residue or residues, for example an alanine residue, precedes the N-terminal residue of the TIMP-3 polypeptide; or wherein the residue corresponding to Threonine2 of TIMP-3 is mutated to Glycine. Such a mutant is considered to retain activity as an inhibitor of ADAMs, such as TACE, ADAMTS-4 and ADAMTS-5, but to have reduced activity as an inhibitor of MMPs.

### COMPOUNDS

The present invention relates to inhibitors of disintegrin-metalloproteinases (ADAMs), particularly of ADAM17/TACE (tumor necrosis factor α-converting enzyme) and aggrecanases, particularly ADAMTS-4 and ADAMTS-5.

Two families of Zn-endopeptidases, the matrix metalloproteinases (MMPs¹) and disintegrin-metalloproteinases (ADAMs), catalyze important proteolytic reactions in the extracellular matrix (ECM) and at the cell surface. The turnover of proteins in the matrix, catalyzed principally by MMPs, is necessary for morphogenesis, tissue remodeling, blastocyst implantation, wound healing and many other important physiological processes (1), while ADAMs catalyze the shedding of the ectodomains of cell surface proteins, releasing cytokines, growth factors, cell adhesion molecules and receptors (2, 3), processes linked to signal transduction, cell growth, cell-cell and cell-matrix interactions. Enhanced activities of specific MMPs and ADAMs underlie or contribute to many critical human diseases including cancer, rheumatoid arthritis, osteoarthritis and heart disease (1-3).

MMP activities in the extracellular matrix are regulated by four endogenous inhibitory proteins, tissue inhibitors of metallo-proteinases (TIMPs)-1 to -4. These are, with few exceptions, broad-spectrum inhibitors of the more than twenty MMPs found in humans (4). In addition, TIMP-3 efficiently inhibits some adamalysins, including ADAM10 (5), ADAM12-S (6), ADAM17/TACE (tumor necrosis factor α-converting enzyme; (7)) and certain ADAMs with thrombospondin motifs, such as ADAMTS-4 and ADAMTS-5 (8); TIMP-1 also inhibits ADAM-10 (5).

TIMPs have two domains and exhibit multiple biological activities such as the stimulation of the growth of certain cells, induction or protection from apoptosis and inhibition of angiogenesis (9, 10). The metalloproteinase inhibitory activity resides in the larger (˜120-residue) N-terminal domain whereas the smaller, ˜65-residue, C-terminal domain mediates interactions with the hemopexin domains of some pro-MMPs. Mutations in the human TIMP-3 gene that result in X to Cys substitutions and truncations in the C-terminal domain of human TIMP-3 are the cause of Sorsbys fundus dystrophy, an autosomal dominant disorder that produces early onset macular degeneration (11, 12).

The structures of complexes of TIMP-1 with the catalytic domain of MMP-3 (13) and of TIMP-2 with a membrane type MMP, MMP-14 (MT1-MMP; (14)), show that a structurally contiguous region around the conserved Cys¹ to Cys⁷⁰ disulfide bond of TIMP (TIMP-1 sequence numbering) inserts into the active site groove of the MMP. Cys¹ bidentally coordinates the catalytic Zn²⁺ through its α-amino and carbonyl groups while the side chain of residue 2 (Thr or Ser) enters into the mouth of the S1′ specificity pocket of the protease. Most (75%) of the interactions with the MMP involve two sections of polypeptide chain of the TIMP around the Cys¹ to Cys⁷⁰ disulfide bond (residues 1-4 and 66-70, see FIG. 1). Blocking the N-terminal α-amino group by carbamoylation (15) or acetylation (16), as well as addition of an extra residue (16, 17) inactivates MMP inhibitory activity of TIMPs. Substitutions for key amino acids in the interaction interface, residues 2, 4 or 68, singly and in combination, differentially affect the affinity of N-TIMP-1 for different MMPs (18, 19). This suggests that the specificity of TIMPs can be modified to produce more targeted MMP inhibitors.

TACE (ADAM-17) is a type-1 membrane protein composed of an extracellular multi-domain region, a transmembrane segment and a C-terminal cytoplasmic domain. Within the extracellular region of the active enzyme are a metalloendopepeptidase catalytic domain, a disintegrin domain, a cysteine-rich domain and a crambin-like domain (2, 3). Many previous studies of the structural, catalytic and inhibitory properties of TACE have focused on the truncated catalytic domain (20-24) but some studies suggest that the non-catalytic domains of the extracellular region have a significant influence on the enzymatic properties such as substrate recognition and zymogen activation (25, 26).

Some ADAMs lack protease activity, but those that are catalytically active share with the MMPs a canonical Zn-binding HExxHxxGxxH sequence motif and a Met-turn in their catalytic domains (http://www.people.virginia.edu/˜jw7g/). However the ADAMs and MMPs are very divergent in overall sequence and their catalytic domains differ considerably in three dimensional structure (20).

We provide mutants of N-TIMP-3 that are inhibitors of ADAMs, for example TACE, ADAMTS-4, ADAMTS-5 and also ADAM10 and ADAM12-S, but in which the interaction interface for MMPs is disrupted. The properties of such mutants as inhibitors of ADAMs such as TACE and ADAMTS-4 and ADAMTS-5 suggest that the interaction of TIMP-3 with ADAMs such as TACE and ADAMTS-4 and ADAMTS-5 and the mechanism of inhibition are distinct from those for MMPs, and also indicates that such mutants are useful as selective inhibitors of ADAMs such as TACE and ADAMTS-4 and ADAMTS-5. Such mutants are also lead compounds useful in the generation of further selective inhibitors of ADAMs such as TACE, ADAMTS-4 and ADAMTS-5.

A first aspect of the invention provides a mutant TIMP-3 (Tissue Inhibitor of MetalloProteinase-3) polypeptide wherein an additional residue, or 1 up to 2, 3, 4, 5, 6, 8, 10, 12, 15, 18 or 20 residues, lies immediately on the amino-terminal side of the first amino acid residue (Cys1) of the mature TIMP-3 polypeptide; or wherein the residue corresponding to Threonine2 of TIMP-3 is mutated to Glycine, or another of the following L-amino acids: Ala, Cys, Asp, Glu, Phe, His, Ile, Lys, Asn, Pro, Gln, Arg, Val, Trp.

Such mutant TIMP-3 polypeptides are considered to inhibit ADAMs, for example TACE, ADAMTS-4 or ADAMTS-5, but are considered to inhibit MMPs, for example MMP-1, MMP-2, the catalytic domain of stromelysin 1 (MMP-3 (ΔC)) or membrane-type 1 MMP (MMP-14), much more weakly (for example 1, 2 or 3 orders of magnitude less) than, for example, wild-type TIMP-3 or N-TIMP-3.

The additional residue or residues (for example two, three, four or more (up to 20) amino acid residues) is/are located immediately on the N-terminal side of Cysteine1, the first amino acid of the mature, active form of TIMP3. This additional amino acid residue (or further residue or residues) on the amino-terminal side of the N-terminal residue of the TIMP-3 polypeptide may, for example, be an L-Alanine residue or possibly any of the other 19 amino acids that are found naturally in proteins, for example Gly or one of the following L-amino acids: Asp, Cys, Glu, Phe, His, Ile, Lys, Leu, Met, Asn, Pro, Gln, Arg, Ser, Thr, Val, Trp, Tyr.

As discussed in the Examples, an example of a mutant TIMP-3 polypeptide with two amino acid residues located immediately on the N-terminal side of Cysteine1, the first amino acid of the mature, active form of TIMP-3, is a (-2A)N-TIMP-3 mutant, which is considered to be more selective for ADAMTS-5 than N-TIMP-3.

Carbamoylation or acetylation of the N-terminal may also provide a TIMP-3 polypeptide that inhibits ADAMs such as TACE and/or ADAMTS-4 and ADAMTS-5, but inhibits MMPs, for example MMP-1, MMP-2, the catalytic domain of stromelysin 1 (MMP-3 (ΔC)) or membrane-type 1 MMP (MMP-14) much more weakly than wild-type TIMP-3 or N-TIMP-3, but such modifications are considered to be harder to prepare reliably.

The term TIMP-3 is well known in the art. The sequence of human TIMP-3, for example, is given in Accession No NP_{—}000353 (FIG. 4) and TIMP-3 is discussed in, for example, the references cited in that record. The TIMP-3 sequence shown includes a pre-sequence. The mature sequence of TIMP-3 starts with residues CTCSPSH . . . . The polynucleotide sequence of the TIMP-3 gene is given in Accession No NM_{—}000362 (FIG. 5). See also US20030143693, which relates to TIMP-3.

The terms ADAM, TACE, ADAMTS-4 and ADAMTS-5, as well as other classes or individual metalloproteinases referred to herein are also well known in the art, as is apparent, for example, from references cited herein.

The mutant TIMP-3 polypeptide may be a mutant N-TIMP-3 polypeptide with the required mutations. N-TIMP-3 corresponds to residues 1 to 121 of full length TIMP-3. The sequence of human N-TIMP-3 is shown in FIG. 6, taken from Lee et al (2002) Protein Science 11, 2493-2503. N-TIMP-3 is considered to retain the inhibitory properties of full length TIMP-3 but may be easier to refold and otherwise handle than full length TIMP-3. N-TIMP-3 also has a reduced tendency to bind to other proteins of the extracellular matrix, as compared with TIMP-3, increasing its availability as a metalloproteinase inhibitor in tissues in a therapeutic context.

The mutant TIMP-3 polypeptide may comprise a further non-TIMP-3 moiety (for example forming a fusion polypeptide with the mutant TIMP-3 moiety). Such a moiety is typically located at the C-terminus of the mutant TIMP-3 polypeptide and be useful in, for example, purifying the polypeptide, targeting the polypeptide to a specific tissue, detecting the polypeptide or promoting dimer formation. Examples of suitable such further moieties will be well known to those skilled in the art. For example a chitin binding domain or cellulose binding domain may be useful for purification. An IgG Fab domain may be useful in promoting dimerisation. As an example, the mutant TIMP-3 polypeptide may have a His-tag, as well known to those skilled in the art, for example 8 histidines, at the C-terminus. Such a tag allows the mutant TIMP-3 polypeptide to be prepared using a Ni-chelate column, as well known to those skilled in the art.

The mutant TIMP-3 polypeptide may be expressed with a presequence, as well known to those skilled in the art, for example with the TIMP-3 presequence (mtpwlglivllgswslgdwgaea) or with a presequence appropriate for expression in cells from a different organism, for example a yeast, insect or bacterial presequence as appropriate. The presequence may be cleaved off (either by the expressing cell's enzymes or by added enzymes) to yield the mature mutant TIMP-3 polypeptide. The mutant TIMP-3 polypeptide may be expressed with an N-terminal methionine residue preceding the mature mutant TIMP-3 polypeptide sequence; the N-terminal methionine may also be cleaved off by the expressing cell's enzymes. For the “wild-type” protein and the T2G mutant and -1A mutants this appears to be the case, though it is possible that a small fraction is not so cleaved. This is also expected to happen with other T2X mutants but for some other -1X constructs the N-terminal methionine may not be cleaved off.

Suitable expression constructs will be known to the skilled person. For example, an adenovirus vector may be used to deliver TIMP-3 to animals for preclinical tests or to patients. Others such as lentivirus will be useful. A vector containing type II collagen promoter may also be useful to express TIMP-3 in the cartilage.

The mutant TIMP-3 polypeptide may be a non-human TIMP-3 (for example non-human N-TIMP-3) polypeptide with the required mutation. For example, the mutant TIMP-3 polypeptide may be a mutant mouse or other rodent TIMP-3 (for example N-TIMP-3) polypeptide or a mutant chicken TIMP-3 (for example N-TIMP-3) polypeptide. The mutant TIMP-3 polypeptide may differ from a naturally occurring TIMP-3 polypeptide only in the mutations indicated above, or may differ in further respects from the sequence of a naturally occurring TIMP-3 polypeptide, for example may differ (for example by conservative or non-conservative mutation, deletion or insertion) from the naturally occurring TIMP-3 polypeptide in up to an additional 1, 2, 5, 10 or 20% of the residues of the naturally occurring TIMP-3 polypeptide or fragment thereof. The mutant TIMP-3 polypeptide may also, as noted above, be a fusion polypeptide, for example may be Myc epitope-tagged or His-tagged, as well known to those skilled in the art.

It is particularly preferred that the mutant TIMP-3 polypeptide has at least 30%, preferably at least 50%, preferably at least 70% and more preferably at least 90% of the inhibitory activity of human T2G N-TIMP-3 or -1A N-TIMP-3 with respect to human TACE or a soluble form of human TACE (for example TACE R651; see Reference 28 of Example 1), for example as assessed using assays generally as described in the Examples. It is further preferred that the mutant TIMP-3 polypeptide inhibits MMPs, for example MMP-1, MMP-2, the catalytic domain of stromelysin 1 (MMP-3 (ΔC)) or membrane-type 1 MMP (MMP-14), much more weakly (for example 1, 2 or 3 orders of magnitude less) than, for example, wild-type TIMP-3 or N-TIMP-3.

By “conservative substitutions” is intended combinations such as Gly, Ala; Val, Ile, Leu; Asp, Glu; Asn, Gln; Ser, Thr; Lys, Arg; and Phe, Tyr.

The three-letter amino acid code of the IUPAC-IUB Biochemical Nomenclature Commission is used herein, with the exception of the symbol Zaa (negatively charged amino acid). In particular, Xaa represents any amino acid. It is preferred that Xaa and Zaa represent a naturally occurring amino acid. It is preferred that the amino acids are L-amino acids.

Particularly preferred amino acid sequences of the mutant TIMP-3 polypeptides will be apparent to the skilled person from the discussion above and from the Examples, and are also set out in the claims.

It is particularly preferred if the mutant TIMP-3 polypeptide has an amino acid sequence which has at least 65% identity with an amino acid sequence set out in claim 2, more preferably at least 70%, 71%, 72%, 73% or 74%, still more preferably at least 75%, yet still more preferably at least 80%, in further preference at least 85%, in still further preference at least 90% and most preferably at least 95% or 97% identity with the amino acid sequence defined above.

As well known to those skilled in the art, the percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al (1994) Nucl Acid Res 22, 4673-4680). The parameters used may be as follows:

Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alignment of TIMP-3 polypeptide sequences and requirements for TIMP-3 inhibitory activity against TACE are also discussed in, for example, Lee et al (2002) Protein Science 11, 2493-25-3 and Lee et al (2002) Biochem J 364, 227-234.

It is preferred that the mutant TIMP-3 polypeptide (or, as appropriate TACE, ADAMTS-4, ADAMTS-5 or other metalloproteinase) is a polypeptide which consists of the amino acid sequence (mutated as set out in claim 1) of the human TIMP-3 or N-TIMP-3 sequence referred to above or naturally occurring allelic variants thereof. It is preferred that the naturally occurring allelic variants are mammalian, preferably human, but may alternatively be homologues from experimental or domestic animals, for example rodents (for example mice or rats), dogs, cats, horses, ovids (for example sheep or goats) or bovines. Examples of such organisms and homologues will be known to those skilled in the art.

A further aspect of the invention provides a polynucleotide encoding a mutated TIMP-3 polypeptide of the invention. A still further aspect of the invention provides a recombinant polynucleotide suitable for expressing a mutated TIMP-3 polypeptide of the invention. Such a polypeptide may, for example, comprise a polynucleotide having a sequence as set out in claim 4 with, for example the addition of a further 5′ initiation codon (ATG) or other control sequences, as well known to those skilled in the art. A yet further aspect of the invention provides a host cell comprising a polynucleotide of the invention.

A further aspect of the invention provides a method of making a mutated TIMP-3 polypeptide of the invention, the method comprising culturing a host cell of the invention which expresses said mutated TIMP-3 polypeptide and isolating said mutated TIMP-3 polypeptide.

A further aspect of the invention provides a mutated TIMP-3 polypeptide obtainable by the above method.

Examples of these aspects of the invention are provided in Example 1, and may be prepared using routine methods by those skilled in the art.

For example, the above mutated TIMP-3 polypeptide may be made by methods well known in the art and as described below and in Example 1, for example using molecular biology methods or automated chemical peptide synthesis methods.

It will be appreciated that peptidomimetic compounds may also be useful. Thus, by “polypeptide” or “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Mézière et al (1997) J. Immunol. 159, 3230-3237, incorporated herein by reference. This approach involves making pseudopeptides containing changes involving the backbone, and not the orientation of side chains. Retro-inverse peptides, which contain D-amino acids, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity as a peptide bond.

It will be appreciated that the peptide may conveniently be blocked at its N- or C-terminus so as to help reduce susceptibility to exoproteolytic digestion.

The invention further provides a method of identifying a compound that is expected to inhibit an ADAM metalloproteinase (for example TACE, ADAMTS-4 or ADAMTS-5) to a greater extent than an MMP (matrix metalloproteinase), comprising the steps of comparing a structure of a test compound with a structure of at least the N-terminal 4, 5, 6, 7, 8, 9 or 10 amino acids of a mutant TIMP-3 polypeptide of the invention (for example as set out in claim 2); and selecting a compound that is considered to have a structure similar to that of the at least the N-terminal 4, 5, 6, 7, 8, 9 or 10 amino acids of a mutant TIMP-3 polypeptide of the invention.

The structure of the at least the N-terminal 4, 5, 6, 7, 8, 9 or 10 amino acids of a mutant TIMP-3 polypeptide of the invention may be a structure modeled on a N-TIMP-3 model, for example as discussed in Lee et al (2002) Protein Science 11, 2493-2503. The selected compound may be one that is considered, from the structural comparison, to interact with TACE or other ADAM, for example ADAMTS-4 or ADAMTS-5 in a similar way to a mutant TIMP-3 polypeptide of the invention.

When selecting a compound that is expected to inhibit ADAMTS-5, it may be particularly useful to select a compound that is considered to have a structure similar to that of the at least the N-terminal 4, 5, 6, 7, 8, 9 or 10 amino acids of a (-2A) mutant TIMP-3 polypeptide of the invention (i.e. with two alanine residues on the N-terminal side of Cysteine1 of the TIMP-3 sequence).

The three-dimensional structures may be displayed by a computer in a two-dimensional form, for example on a computer screen. The comparison may be performed using such two-dimensional displays.

The following relate to molecular modelling techniques: Blundell et al (1996) Structure-based drug design Nature 384, 23-26; Bohm (1996) Computational tools for structure-based ligand design
Prog Biophys Mol Biol 66(3), 197-210; Cohen et al (1990) J Med Chem 33, 883-894; Navia et al (1992) Curr Opin Struct Biol 2, 202-210.

The following computer programs, for example, may be useful in carrying out the method of this aspect of the invention: GRID (Goodford (1985) J Med Chem 28, 849-857; available from Oxford University, Oxford, UK); MCSS (Miranker et al (1991) Proteins: Structure, Function and Genetics 11, 29-34; available from Molecular Simulations, Burlington, Mass.); AUTODOCK (Goodsell et al (1990) Proteins: Structure, Function and Genetics 8, 195-202; available from Scripps Research Institute, La Jolla, Calif.); DOCK (Kuntz et al (1982) J Mol Biol 161, 269-288; available from the University of California, San Francisco, Calif.); LUDI (Bohm (1992) J Comp Aid Molec Design 6, 61-78; available from Biosym Technologies, San Diego, Calif.); LEGEND (Nishibata et al (1991) Tetrahedron 47, 8985; available from Molecular Simulations, Burlington, Mass.); LeapFrog (available from Tripos Associates, St Louis, Mo.); Gaussian 92, for example revision C (M J Frisch, Gaussian, Inc., Pittsburgh, Pa. ©1992); AMBER, version 4.0 (P A Kollman, University of California at San Francisco, ©1994); QUANTA/CHARMM (Molecular Simulations, Inc., Burlington, Mass. ©1994); and Insight II/Discover (Biosym Technologies Inc., San Diego, Calif. ©1994). Programs may be run on, for example, a Silicon Graphics™ workstation, Indigo²™ or IBM RISC/6000™ workstation model 550.

Several in silico methods could be employed, for example, via a substructure search for new ligands using programmes such as CHEM DRAW or CHEM FINDER. The basic structure of the ligand (for example the mutated TIMP-3 polypeptide) or part thereof capable of binding to the ADAM is taken (or predicted) and various structural features of it are submitted to a programme which will search a set of chemical company catalogues for chemicals containing this substructure.

A starting compound may initially be selected by screening for an inhibitory effect on an ADAM, for example TACE; then compared with the structure; used as the basis for designing further compounds which may then be tested by further modelling and/or synthesis and assessment, as discussed further below.

The selected compounds may then be ordered or synthesised and assessed, for one or more of ability to bind to and/or inhibit ADAM and/or MMP activity.

The method of the invention may further comprise the steps of providing, synthesising, purifying and/or formulating a compound selected using computer modelling, as described above; and of assessing whether the compound inhibits the activity of one or more ADAMs and/or MMPs. The compound may be formulated for pharmaceutical use, for example for use in in vivo trials in animals or humans.

A compound that inhibits the activity of one or more ADAM more than one or more MMP, as discussed above, may be selected.

As noted above, the selected or designed compound may be synthesised (if not already synthesised) or purified and tested for its effect on an ADAM and/or an MMP. The compound may be tested in an in vitro screen for its effect on an ADAM and/or MMP or on a cell or tissue in which an ADAM and/or MMP is present. The cell or tissue may contain an endogenous ADAM and/or MMP and/or may contain an exogenous ADAM and/or MMP (including an ADAM and/or MMP expressed as a result of manipulation of endogenous nucleic acid encoding the ADAM or MMP). The compound may be tested in an ex vivo or in vivo screen, which may use a transgenic animal or tissue. The compound may also be tested, for comparison, in a cell, tissue or organism that does not contain the ADAM or MMP (or contains reduced amounts of the ADAM or MMP), for example due to a knock-out or knock-down of one or more copies of the ADAM or MMP gene. Suitable tests will be apparent to those skilled in the art and examples include assessment of shedding, for example of TNFα, assessment of cartilage degradation, or of synovial cell proliferation in animal models of arthritis, for example collagen type II induced arthritis (CIA).

The ability of the compound to inhibit an ADAM (for example TACE, ADAMTS-4 or ADAMTS-5) or an MMP (for which preferences are also given above) may be assessed using methods well known to those skilled in the art, for example methods such as those described in the Examples. For example enzyme assays using purified components, shedding assays or cartilage aggrecan degradation assays may be used, for example as described in the Examples. WO 2004/006925, for example, also describes assays that may be used in assessing inhibitors of TACE. Protocols which can be used for other expressed and purified pro MMPs using substrates and buffers conditions optimal for the particular MMP are described in, for example C. Graham Knight et al., (1992) FEES Lett. 296 (3): 263-266. The ability of compounds or mutants of this invention to inhibit the cellular processing of TNFa production may be assessed, for example, in THP-1 cells using an ELISA to detect released TNF essentially as described K. M. Mohler et al., (1994) Nature 370: 218-220. The processing or shedding of other membrane molecules such as those described in N. M. Hooper et al., (1997) Biochem. J. 321: 265-279 may, for example, be tested using appropriate cell lines and with suitable antibodies to detect the shed protein. The ability of the mutants or compounds of this invention to inhibit the degradation of the aggrecan or collagen components of cartilage can be assessed, for example, essentially as described by K. M. Bottomley et al., (1997) Biochem J. 323:483-488. The ability of the mutants or compounds of this invention as in vivo TNFa inhibitors can be assessed, for example, in the rat. Briefly, groups of female Wistar Alderley Park (AP) rats (90-100 g) are dosed with compound (5 rats) or drug vehicle (5 rats) by the appropriate route e.g. peroral (p. o.), intraperitoneal (i. p.), subcutaneous (s. c.) 1 hour prior to lipopolysaccharide (LPS) challenge (30 gg/rat i. v.). Sixty minutes following LPS challenge rats are anaesthetised and a terminal blood sample taken via the posterior vena cavae. Blood is allowed to clot at room temperature for 2 hours and serum samples obtained. These are stored at −20° C. for TNFa ELISA and compound concentration analysis. Data analysis by dedicated software calculates for each compound/dose: Percent inhibition of TNFa=Mean TNFa (Vehicle control)−Mean TNFa (Treated)×100 Mean TNFa (Vehicle control). Activity of a compound as an anti-arthritic can, for example, be tested in the collagen-induced arthritis (CIA) as defined by D. E. Trentham et al., (1977) J. Exp. Med. 146: 857. In this model acid soluble native typeII collagen causes polyarthritis in rats when administered in Freunds incomplete adjuvant. Similar conditions can be used to induce arthritis in, for example, mice.

Compounds may also be subjected to other tests, for example toxicology or metabolism tests, as is well known to those skilled in the art.

The tested compounds may be, for example, peptidomimetic compounds or antibodies. By the term “antibody” is included synthetic antibodies and fragments and variants (for example humanised or other mutated antibody molecules, as known to those skilled in the art) of whole antibodies which retain the antigen binding site. The antibody may be a monoclonal antibody, but may also be a polyclonal antibody preparation, a part or parts thereof (for example an Fab fragment or F(ab′)₂) or a synthetic antibody or part thereof. Fab, Fv, ScFv and dAb antibody fragments can all be expressed in and secreted from E. coli, thus allowing the facile production of large amounts of the said fragments. By “ScFv molecules” is meant molecules wherein the V_{H} and V_{L} partner domains are linked via a flexible oligopeptide. IgG class antibodies are preferred.

Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: techniques and Applications”, J G R Hurrell (CRC Press, 1982), modified as indicated above. Phage display-based techniques may alternatively be used, as well known to those skilled in the art. Bispecific antibodies may be prepared by cell fusion, by reassociation of monovalent fragments or by chemical cross-linking of whole antibodies. Methods for preparing bispecific antibodies are disclosed in Corvalen et al, (1987) Cancer Immunol. Immunother. 24, 127-132 and 133-137 and 138-143.

A general review of the techniques involved in the synthesis of antibody fragments which retain their specific binding sites is to be found in Winter & Milstein (1991) Nature 349, 293-299.

The compounds identified in the methods may themselves be useful as a drug or they may represent lead compounds for the design and synthesis of more efficacious compounds.

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons. A drug-like compound may additionally exhibit features of selective interaction with a particular protein or proteins and be bioavailable and/or able to penetrate cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

It is appreciated that screening assays which are capable of high throughput operation are particularly preferred.

It will be understood that it will be desirable to identify compounds or mutants that may modulate the activity of the ADAM, for example TACE, in vivo. Thus it will be understood that reagents and conditions used in the method may be chosen such that the interactions between, for example, the ADAM and the compound or mutant, are substantially the same as between the human ADAM and the compound or mutant in vivo.

A still further aspect of the invention is a polypeptide or polynucleotide of the invention (or a compound identified or identifiable by the above selection/design methods of the invention), for use in medicine. Conditions or diseases in which such compounds, polypeptides or polynucleotides may be useful are indicated below.

The polypeptide, polynucleotide or compound may be administered in any suitable way, usually parenterally, for example intravenously, intraperitoneally or intravesically, in standard sterile, non-pyrogenic formulations of diluents and carriers. The compound (or polypeptide or polynucleotide) may also be administered topically, which may be of particular benefit for treatment of surface wounds. The compound (or polypeptide or polynucleotide) may also be administered in a localised manner, for example by injection.

A further aspect of the invention provides the use of a polypeptide or polynucleotide (or compound) of the invention in the manufacture of a medicament for the treatment of a patient in need of inhibition of one or more ADAMs, for example TACE (TNFα Converting Enzyme), ADAMTS-4 or ADAMTS-5

The patient may be a patient with an inflammatory disease that involves unregulated or dysregulated shedding of TNF-αc. TACE activity has also been implicated in the shedding of other membrane bound proteins including TGFa, p75 & p55 TNF receptors, L-selectin and amyloid precursor protein [Black (2002) Int. J. Biochem. Cell Biol. 34:1-5]. In view of this, the patient may be a patient with rheumatoid arthritis or osteoarthritis. The patient may be a patient with rheumatoid arthritis or osteoarthritis, including initial stages of the disease diagnosed radiologically or using other methods, or unregulated breakdown of articular cartilage, which ADAMTS-4 and ADAMTS-5 are considered to be involved in. ADAMTS-4 and ADAMTS-5 degrade aggrecan, fibromodulin, decorin and biglycan.

Thus, a further aspect of the invention provides the use of a polypeptide or polynucleotide (or compound) of the invention in the manufacture of a medicament for treating rheumatoid arthritis, osteoarthritis, osteopenia, osteolysis, osteoporosis, psoriasis, Crohn's disease, ulcerative colitis, multiple sclerosis, degenerative cartilage loss, sepsis, septic shock, AIDS, HIV infection [Peterson, P. K.; Gekker, G.; et. al. J. Clin. Invest. 1992, 89, 574; Pallares-Trujillo, J.; Lopez-Soriano, F. J. Argiles, J. M. Med. Res. Reviews, 1995, 15(6), 533.], graft rejection [Piguet, P. F.; Grau, G. E.; et. al. J. Exp. Med. 1987, 166, 1280.], cachexia [Beutler, B.; Cerami, A. Ann. Rev. Biochem. 1988, 57, 505.], anorexia, inflammation [Ksontini, R.; MacKay, S. L. D.; Moldawer, L. L. Arch Surg. 1998, 133, 558.], abdominal aortic aneurysm, stroke, congestive heart failure [Packer, M. Circulation, 1995, 92(6), 1379; Ferrari, R.; Bachetti, T.; et. al. Circulation, 1995, 92(6), 1479.], post-ischaemic reperfusion injury, inflammatory disease of the central nervous system, inflammatory bowel disease or insulin resistance [Hotamisligil, G. S.; Shargill, N. S.; Spiegelman, B. M.; et. al. Science, 1993, 259, 87.]. These diseases or conditions are considered to be linked with excess activity of TACE, ADAMTS-4, ADAMTS-5 and possibly ADAM-10.

These conditions are considered to be examples of conditions or diseases mediated by TNFα. Use of a polypeptide or polynucleotide (or compound) of the invention in the manufacture of a medicament for treating other such conditions or diseases is also included within the scope of the present invention. An inhibitor of TACE and/or of ADAM-10 (also considered to have a role in TNF-alpha shedding) that inhibits to a lesser extent MMPs is considered to be useful in the treatment or prophylaxis of these conditions. Conditions mediated by TNFα are well known to the skilled person and discussed extensively, for example in US 2005113346, “TNF-[alpha] in Human Diseases”, Current Pharmaceutical Design, 1996, 2, 662; WO 2004/006925; US2005075384, which mentions septic shock, haemodynamic shock, sepsis syndrome, post ischemic reperfusion injury, malaria, Crohn's disease, inflammatory bowel diseases, mycobacterial infection, meningitis, psoriasis, congestive heart failure, fibrotic diseases, cachexia, graft rejection, cancer, diseases involving angiogenesis, autoimmune diseases, skin inflammatory diseases, osteoarthritis, rheumatoid arthritis, multiple sclerosis, radiation damage, hyperoxic alveolar injury, periodontal disease, HIV and non-insulin dependent diabetes mellitus; U.S. Pat. No. 6,534,475, which mentions neovascularization, rubeosis iridis, neovascular glaucoma, age-related macular degeneration, diabetic retinopathy, ischemic retinopathy, and retinopathy of prematurity.

As a prophylactic treatment, inhibition of ADAMTS-4 or ADAMTS-5 may be particularly useful, for example with osteoarthritis. These enzymes are considered to act on cartilage over a period of many years. The process underlying the disease is considered to take from 10-30 years. Thus, prophylactic treatment may be desirable in those considered to be at risk of developing the disease, or those with very early stages of the disease.

A further aspect of the invention provides a method of treating a patient in need of inhibition of one or more ADAMs, for example TACE (TNFα Converting Enzyme), ADAMTS4 or ADAMTS5, comprising administering to the patient a therapeutically effective amount of a polypeptide or polynucleotide (or compound) of the invention.

All documents referred to herein are hereby incorporated by reference.

The invention is now described in more detail by reference to the following, non-limiting, Figures and Examples.

### FIGURE LEGENDS

FIG. 1. Structural model of the core region of the reactive site of TIMP-3. The image was produced from a model of a complex of N-TIMP-3 with MMP-3, which was derived from the crystal structure of TIMP-1/MMP-3 complex (pdb file 1UEA; (13)) and a modelled structure for human TIMP-3 in the SWISS-MODEL repository (48). The C-terminal domains of both TIMPs were removed by text editing. The N-TIMP-3 structure was superimposed on the coordinates of N-TIMP-1 in 1UEA, and adjusted manually to ensure that the N-terminal four residues of the two structures are precisely superimposed. This was carried out and the image was generated using the UCSF Chimera package from the Computer Graphics Laboratory, University of California, San Francisco (supported by NIH P41 RR-04081; (49)).

FIG. 2. Inhibition of MMP and TACE by N-TIMP-3 and its mutants. A. Inhibition of MMP-14(CD) by wild-type and mutated N-TIMP-3. Open circles, wild-type N-TIMP-3; closed circles, T2G; and open squares, -1A. B. Comparison of the inhibition of TACE by wild-type N-TIMP-3, N-TIMP-1 and TAPI-2. The inhibitors were incubated with 0.5 nM TACE for 3 hr at room temperature, and the residual enzyme activity was measured with 10 μM Substrate III (R&D Systems). The assays were performed at pH 9.0 at a final NaCl concentration of 1 mM. Open circles, N-TIMP-3; closed circles, TAPI-2; and open squares, N-TIMP-1. C. Inhibition of TACE (0.5 nM) by wild-type and mutated N-TIMP-3. Open circles, wild-type inhibitor; closed circles, T2G; and open squares, -1A.

FIG. 3. Effects of mutations in N-TIMP-3 on inhibition of cellular shedding of TNF-α. THP-1 cells (2.5×10⁶/ml) growing in serum-free RPMI-1640 medium were stimulated with 100 ng/ml PMA for 20 min before adding various concentrations of N-TIMP-3 (wild-type and mutants). Cells were allowed to grow for another 6 hr and conditioned media were collected for the ELISA assays.

FIG. 4. Sequence of TIMP-3 with Presequence

FIG. 5. Sequences Encoding Mutant TIMP-3 and N-TIMP-3 Polypeptides

The sequences include an ATG initiation codon (Met), all possible codons for the mutated amino acid or acids and a termination codon (italicized).

FIG. 6. Inhibition of ADAMTS-4 by N-TIMP-3 Mutants

ADAMTS-4 lacking the spacer domain (0.5 nM) was incubated with N-TIMP-3 mutants at the concentration indicated for 30 min and then incubated with 1 mg/ml of bovine aggrecan at pH 7.5 for 2 h at 37° C. The reaction was terminated with 10 mM EDTA and samples were deglycosylated and subjected to Western blotting analysis using antibodies that recognise the fragments with the C-terminal GELE1480 as described by Little et al [17]. The bands were quantified by densitometric analyses.

FIG. 7. Inhibition of IL-1a stimulated porcine articular cartilage degradation by N-terminal mutants of N-TIMP-3. Porcine articular cartilage pieces were cultured for three days. Cartilage was stimulated with IL-1α (10 ng/ml) with TIMPs at the concentrations indicated. Glycosaminoglycan (GAG) release in the media was measured by dimethyl methylene blue (DMMB). N-TIMP-3 and the N-terminal mutants dose dependently inhibited degradation whereas TIMP-1 and TIMP-2 did not.

FIG. 8. Graphs of Kᵢ₍ₐₚₚ₎ determination. The GST-IGD-FLAG substrate assay was used to determine the Kᵢ₍ₐₚₚ₎ of the N-terminal reactive site mutants against ADAMTS-4 (filled squares) and ADAMTS-5 (open circles).

FIG. 9. The effect of TIMP-3 mutants on TNFα release by monocyte-derived-macrophages (MDM). MDM derived from a normal subject were incubated with increasing concentrations of the TIMP-3 mutant protein in the presence of 10 ng/ml LPS. Data is normalised to % LPS stimulation.

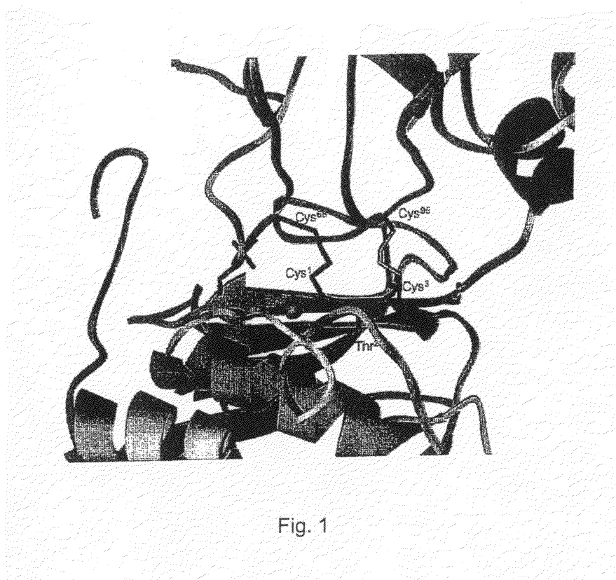

### EXAMPLE 1

### Reactive Site Mutations in Tissue Inhibitor of Metalloproteinase-3 Disrupt Inhibition of Matrix Metalloproteinases but not TNF-α Converting Enzyme

Tissue inhibitor of metallo-proteinase-3 (TIMP-3) is a dual inhibitor of the matrix metalloproteinases (MMPs) and some ADAMs (adamalysins), two families of extracellular and cell surface metallo-proteinases that function in extracellular matrix turnover and the shedding of cell surface proteins. The mechanism of inhibition of MMPs by TIMPs has been well characterized and, since the catalytic domains of MMPs and adamalysins are homologous, it was assumed that the interaction of TIMP-3 with adamalysins is closely similar. Here we report that the inhibition of the extracellular region of ADAM-17 (TACE) by the inhibitory domain of TIMP-3 (N-TIMP-3) shows positive cooperativity. Also, mutations in the core of the MMP-interaction surface of N-TIMP-3 dramatically reduce the binding affinity for MMPs, but have little effect on the inhibitory activity for TACE. These results suggest that the mechanism of inhibition of ADAM-17 by TIMP-3 may be distinct from that for MMPs. The mutant proteins are also effective inhibitors of TNF-α release from phorbol ester-stimulated cells, indicating that they provide a lead for engineering TACE-specific inhibitors that may reduce side effects arising from MMP inhibition and are possibly useful for treatment of such diseases associated with excessive TACE activity as rheumatoid arthritis.

The abbreviations used are: MMP, matrix metalloproteinase; TIMP, tissue inhibitor of metalloproteinase; N-TIMP, N-terminal inhibitory domain of TIMP; ADAM, a disintegrin and metalloproteinase; TACE, tumor necrosis factor α converting enzyme; MT1-MMP, membrane-type metalloproteinase-1; TAPI-2, HONHCOCH₂CH(CH₂CH(CH₃)₂)—CO-t-Butyl-Gly-Ala-NHCH₂CH₂NH₂; Kᵢ^{(app)}, apparent inhibition constant.

### Experimental Procedures

Materials—The plasmid pET-42b-N-timp-3His₈ containing the gene encoding a C-terminally His-tagged form of the N-terminal domain of TIMP-3 in the pET-42b vector (Novagen) was generated as described previously (8). All reagents, cells and instruments used for plasmid construction, and for the expression, purification and in vitro folding of N-TIMP-3 mutants were from the same sources as in previous studies (8). Metalloproteinases and substrates used in the kinetic assays were obtained from previously reported sources (19, 27). N-TIMP-1 was expressed in E. coli and folded in vitro as described (19), and the synthetic metalloproteinase inhibitor TAPI-2 [HONHCOCH₂CH(CH₂CH(CH₃)₂)—CO-t-Butyl-Gly-Ala-NHCH₂CH₂NH₂] was from Peptides International. Human monocyte THP-1 cells and RPMI-1640 medium were purchased from ATCC, while phorbol 12-myristate 13-acetate (PMA) was from Sigma and the antibodies used for ELISA were from BD Pharmingen.

Construction of N-TIMP-3 mutants—The plasmid pET-42b-N-timp-3His₈ was used as the template for site-directed mutagenesis by PCR. The forward primers used (the mutated codons are underlined and the restriction sites are shown in italic) were

5′-AAAACATATGTGCGGATGCTCGCCC-AGCCAC-3′ (for T2G)
and
5′-AAAACATATGGCATGCACATGCTCG-CCCAGCCAC-3′
(for-1Ala).

The reverse primer was

5′-AAAAGCGGCCGCGTTACAACCCA-GGTGATA-3′.

Reactions were carried out for 35 cycles at 94° C. for 1 min, 60° C. for 1 min, and 72° C. for 2 min after a hot start at 94° C. for 3 min in a PCR Sprint HYBAID system using the Vent PCR kit (New England Biolabs). PCR products were cloned back into the pET-42b vector using the NdeI and NotI sites (both enzymes were from New England Biolabs) and confirmed by automatic DNA sequencing using T7 promoter primer.

Expression, purification and in vitro folding of N-TIMP-3 and mutants—N-TIMP-3 and its mutants were expressed in E. coli BL21(DE3) cells as inclusion bodies. The proteins were extracted with 6 M guanidine-HCl and purified by Ni²⁺-chelate chromatography in 6 M guanidine as described previously (8). Purified proteins were treated with cystamine and were folded in vitro by removing the denaturant by dialysis in the presence of 5 mM β-mercaptoethanol and 1 mM 2-hydroxyethyl disulfide essentially as described (8) except that 1 M NaCl was included to enhance protein solubility during the folding process. The folded proteins were subsequently loaded to a 5 ml Ni²⁺-NTA column previously equilibrated with 20 mM Tris-HCl (pH 7.0), 1 M NaCl and 20% glycerol, and eluted with the same buffer containing 200 mM imidazole.

Enzyme inhibition kinetic studies—Inhibition kinetic studies for MMPs and TACE were carried out as described previously (19, 27) with modifications. Purified N-TIMP-3 and mutants were dialyzed against 20 mM Tris-HCl (pH 7.0), 50 mM NaCl containing 20% glycerol, centrifuged at 14,000 rpm for 10 min to remove any precipitate, and protein concentration was re-measured before conducting inhibition assays. Since NaCl inhibits the activity of the TACE ectodomain in vitro (28), we adjusted the final concentration of NaCl to 1 mM in all assays with TACE. Equal volumes (10% of total assay volume) of diluted solutions of N-TIMP-3 and mutants were added to TACE assays, resulting in a final pH of 8.8.

Inhibition data were analyzed by fitting to the following equations as appropriate:

(Eq. 1) Tight binding inhibition:

$$v / {v}_{0} = \frac{E - I - K + {( {( E - I - K )}^{2} + 4 EK )}^{0.5}}{2 E}$$

### (Eq. 2) Normal Inhibition:

v=v₀/(1+I/K)

### (Eq. 3) Cooperative Inhibition (29):

v=v₀/(1+(I/K)^{h})

where v is the experimentally determined reaction velocity, v₀ is the uninhibited activity, E is enzyme concentration, I is inhibitor concentration, K is the apparent inhibition constant (Kᵢ^{(app)}) and h is the Hill coefficient.

Inhibition of TNF-α shedding from THP-1 cells—All TIMP solutions were dialyzed against 20 mM Tris-HCl (pH 7.0), 150 mM NaCl and 20% glycerol before use. Human monocyte THP-1 cells cultured in RPMI-1640 medium supplemented with 5% fetal calf serum were harvested, extensively washed and reseeded into serum-free medium at 2.5×10⁶ cells/ml. Shedding was stimulated by adding PMA to a final concentration of 100 ng/ml, and cells were incubated at 37° C. with 5% CO₂ for 20 min before adding 1/10 volume of various concentrations of N-TIMP-3 or a mutant. The cells were then further cultured for another 6 h, and the conditioned media were collected by centrifugation at 3000 rpm. The amount of soluble TNF-α released into the medium was measured using sandwich enzyme-linked immunosorbent assay, as described by Engelberts et al. (30) with modifications. The released TNF-α was absorbed to microtiter plates coated with mouse monoclonal anti-human TNF-α antibody BD551220 (1:200 dilution), and the bound TNF-α was detected using biotinylated mouse monoclonal anti-human TNF-α: antibody BD554511 (1:500 dilution) and streptavidin conjugated with horse radish peroxidase, and 3,3′,5,5′-tetramethylbenzidine as peroxidase substrate (KPL, Guildford, UK). The plates were read at 450 nm with an ELX808 plate reader (BIO-TEK Instruments Inc). The standard curve of recombinant human TNF-α covered the range of 60-5,000 pg/ml.

### Results

Design and production of N-TIMP-3 mutants—Mutations in N-TIMP-3 were designed to disrupt inhibitory activity towards MMPs based on the known structures of TIMP-1/MMP-3 complex and TIMP-2/MT1-MMP complex (13, 14), and previous mutational studies with TIMPs (17, 18). The specific mutations are:

The addition of an N-terminal alanine extension (-1A) to perturb the interaction of Cys¹ with the active site Zn²⁺; this mutation in N-TIMP-1 (our unpublished data) and TIMP-2 (17) drastically curtailed inhibitory activity for MMPs.

A Thr² to Gly (T2G) mutation which removes the side chain of residue 2; this residue interacts with the S1′ specificity pocket of MMPs and this mutation in N-TIMP-1 reduces the affinity for MMPs-1, -2 and -3 about 1000-fold (18).

These mutants, as well as wild-type inhibitor, were expressed in bacteria as inclusion bodies, purified and folded in vitro. A high salt concentration was found to increase the solubility of N-TIMP-3; therefore we included 1 M NaCl throughout the in vitro folding procedure. This significantly increased the yield of N-TIMP-3 and mutants (data not shown).

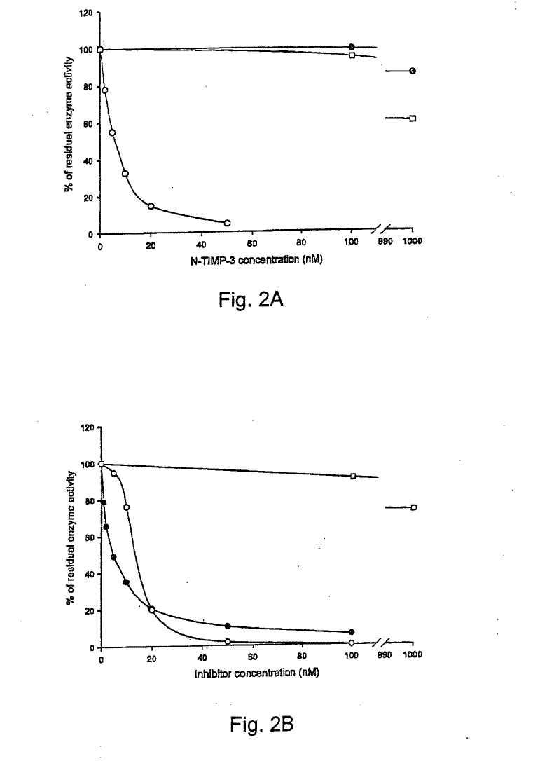

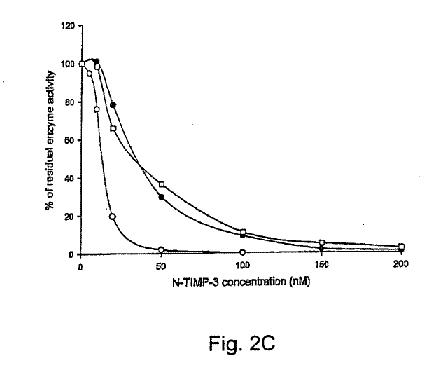

Inhibitory properties of mutants with purified metalloproteinases—The inhibitory activities of wild-type N-TIMP-3 and the two mutants were determined with MMPs representing four different sub-groups: full-length collagenase 1 (MMP-1), gelatinase A (MMP-2), and the catalytic domains of stromelysin 1 (MMP-3(ΔC)) and membrane-type 1 MMP (MMP-14). As previously reported for the corresponding mutants of N-TIMP-1 and TIMP-2 (17, 18), both mutations in N-TIMP-3 reduced the inhibitory activity towards the four MMPs by 2 to 3 orders of magnitude (Table I). FIG. 2A highlights the difference in inhibition of MMP-14(CD) by wild-type and mutated N-TIMP-3.

The inhibitory activities of the mutants were also compared with that of wild-type N-TIMP-3 against a soluble form of TACE in which the transmembrane and C-terminal cytoplasmic domains are deleted (TACE R651; (28)). These assays were carried out at pH 9.0 and low ionic strength, because the activity of TACE is optimal at higher pH ((7) and the protocol from R&D Systems), and is strongly inhibited by salt (28). Both wild-type N-TIMP-3 and the hydroxamate-based inhibitor, TAPI-2, effectively inhibited the activity of TACE; in contrast, wild-type N-TIMP-1 had minimal inhibitory activity under the same condition (FIG. 2B). The inhibition curve of TACE by wild-type N-TIMP-3 is sigmoid, in striking contrast with the inhibition by TAPI-2 and with the inhibition of MMPs by N-TIMP-3 and N-TIMP-1 (FIGS. 2A, 2B; (31)). Sigmoid inhibition curves were also obtained for TACE with the T2G and -1A mutants of N-TIMP-3 (FIG. 2C). These mutations, which severely reduced activity against MMPs, had little effect on the inhibition of TACE. The inhibition data obtained with N-TIMP-3 and its mutants did not fit well with equations 1 or 2 for tight binding or weak to moderate inhibitors, or to other equations describing multi-site binding (not shown), but fit well to equation 3 for positively cooperative binding. The results indicate that the mutations have only a minor effect on the apparent inhibition constant (Kᵢ^{(app)}) but also reduce the Hill coefficient, h (Table II).

The conditions used for TACE and MMP activity measurements differ in pH and ionic strength. To determine if this could influence the inhibitory activities of N-TIMP-3 and mutants, the inhibitory activities of wild-type N-TIMP-3 and the T2G mutant against TACE were also determined at pH 7.5, since MMPs inhibition measurements were conducted at this pH. Sigmoid inhibition curves were obtained for both proteins and Ki values of 26±3 and 46±2 nM, respectively (data not shown). It was not possible to conduct TACE activity measurements at higher NaCl concentrations because of strong enzyme inhibition. To determine if the inhibitory patterns of N-TIMP-3 and mutants are affected by pH and ionic strength, we investigated the inhibition of MMP-1 by N-TIMP-3 and the -1A mutant under the conditions used for TACE activity measurements. Both showed normal hyperbolic inhibition profiles with Ki values of 1.6 nM and 412 nM, respectively (data not shown). Thus, binding of the wild-type inhibitor was not significantly affected at the higher pH and the mutation also strongly disrupts binding, albeit to a 3-fold lower extent than at pH 7.5.

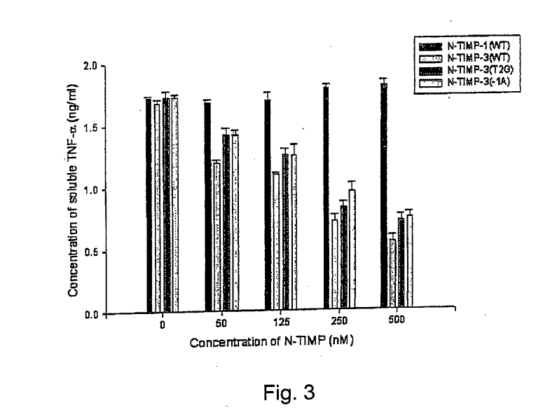

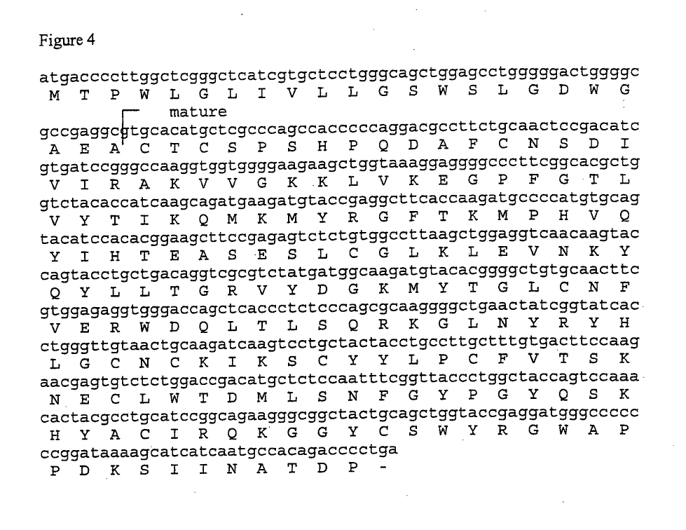

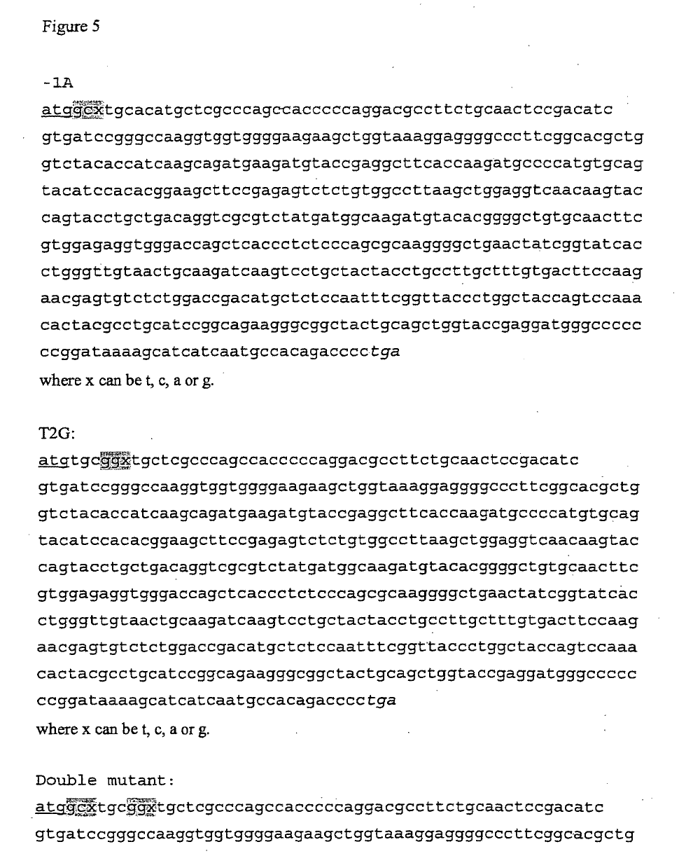

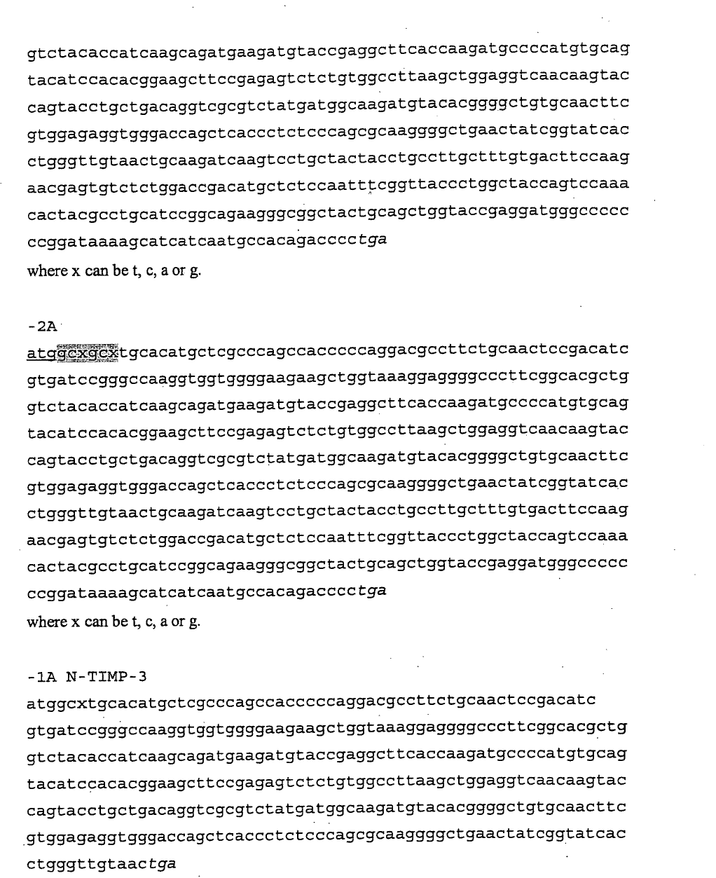

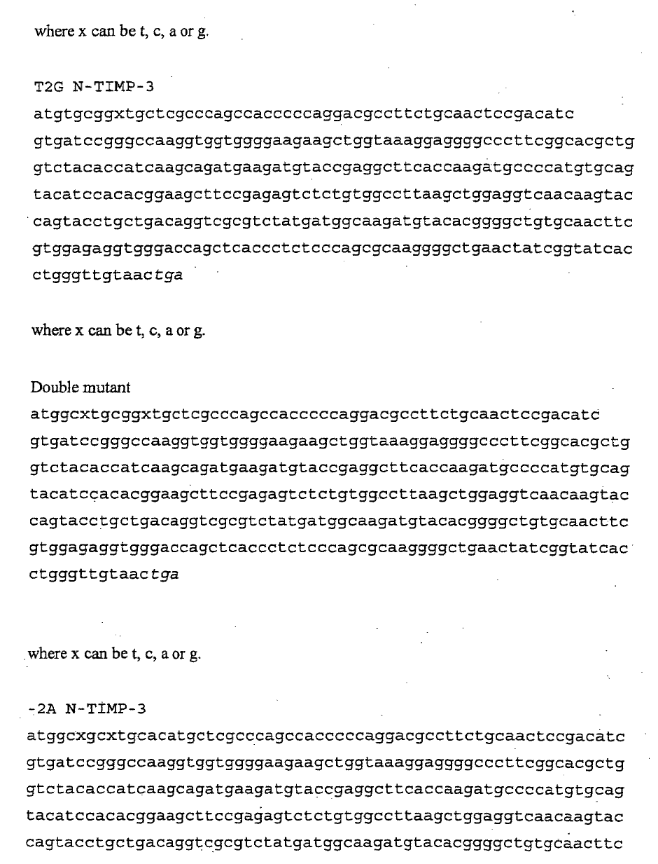

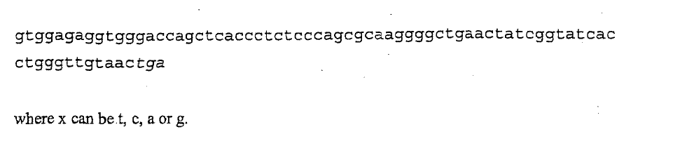

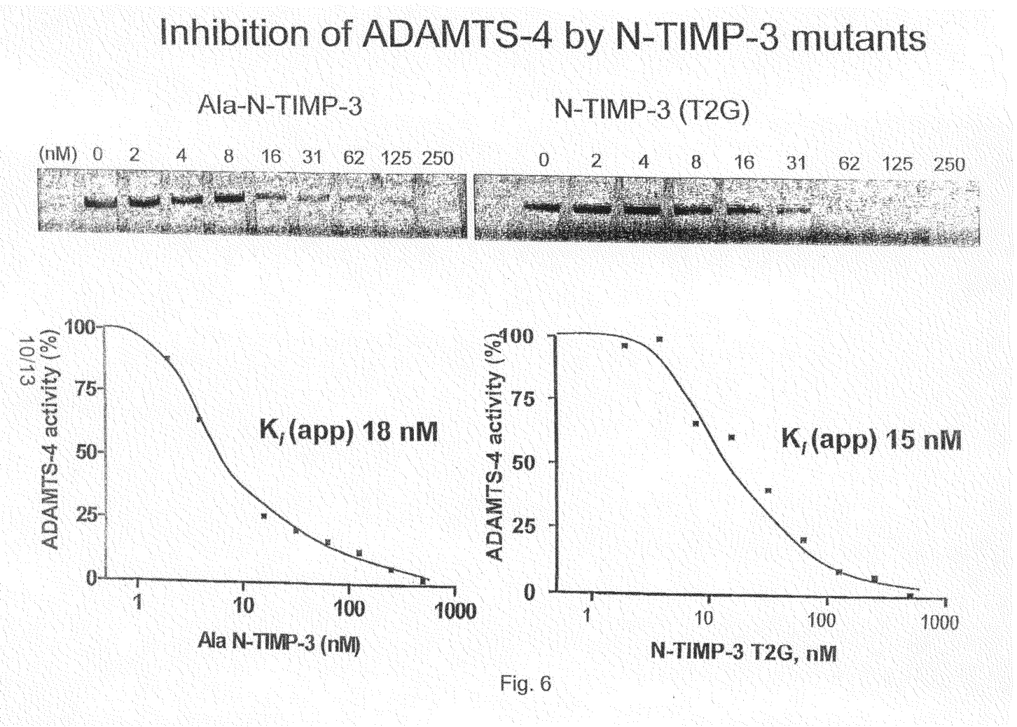

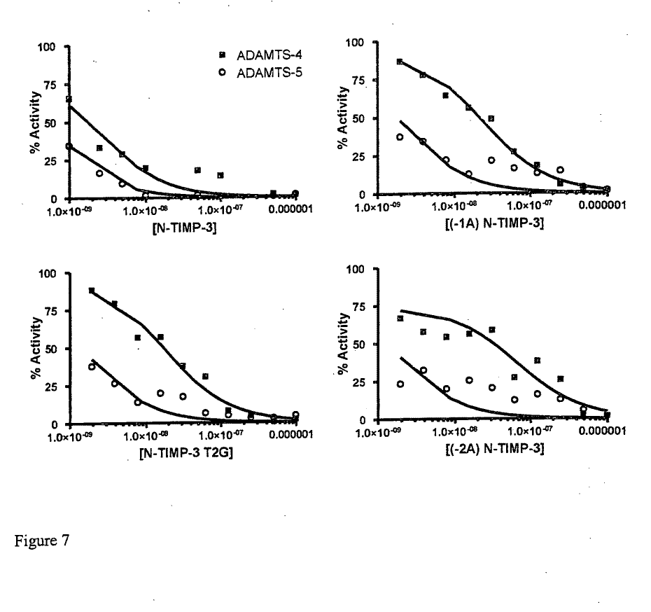

Effects of mutations in N-TIMP-3 on inhibition of cellular shedding of TNF-α—The ectodomains of many cell surface proteins are released in soluble forms through processing catalyzed by cell surface “sheddases”. Both TACE/ADAM17 and ADAM10 have been found to be active as sheddases, TACE being particularly important for the release of the cytokine TNF-α from its cell surface precursor (32). The release of TNF-α from monocytes is a key for inflammation and immunity, making TACE an interesting target for anti-proteolytic therapies. We investigated the abilities of N-TIMP-3 and mutants to inhibit TNF-α shedding from human monocyte THP-1 cells, where TACE, but not other sheddases, was shown to be the major enzyme responsible for releasing TNF-α from cell surface (33). In cell culture systems, higher inhibitor concentrations are required than for the inhibition of purified enzyme in vitro; nevertheless N-TIMP-3, at concentrations of 50 to 500 nM, effectively inhibited the PMA-stimulated release of TNF-α whereas N-TIMP-1 had no effect. As in the studies with pure enzyme shown in FIG. 2C, the T2G and -1A mutations in N-TIMP-3 exhibited only slightly reduced inhibitory activity for TNF-α release (FIG. 3).

### Discussion

Among the four mammalian TIMPs, TIMP-3 has the broadest range as a metalloproteinase inhibitor that includes both the MMPs and disintegrin-metalloproteinases. The latter are complex multi-domain enzymes that share only catalytic and pro-domains with the MMPs. Although the ADAM and MMP catalytic domains are homologous, their levels of sequence identity are low and the crystallographic structure of the TACE catalytic domain indicates that they differ in tertiary structure (20); the rms deviation of ˜120 Cα atoms that are topologically equivalent between the TACE and MMP structures is 1.6 Å. ADAMs have unique structural features including an additional α-helix and a multiple-turn loop, but lack the structural zinc and calcium ions shared by the MMPs (20). Although TACE and MMPs have generally similar active site structures, that of TACE differs in having a deep S3′ pocket merging with the hydrophobic S1′ specificity pocket. Much previous work has focused on the truncated catalytic domain of TACE including structural studies (20) and inhibitory studies using N-TIMPs and their mutants (21-24). In the absence of a structure of a TIMP-3/TACE complex, Lee et al. (34) modeled the structure of TIMP-3 using the known structures of TIMP-1 and TIMP-2 and were able to dock this with the catalytic domain of TACE in a manner similar to that in the two known inhibitory TIMP/MMP complexes. This suggests that the mechanism of TIMP-3 inhibition of TACE could be similar to that for MMPs. However, there is a significant difference in susceptibility to TIMP-3 inhibition between the truncated catalytic domain of TACE and longer forms similar to that used here that contain the disintegrin, cysteine-rich and the crambin-like domains (35). Non-catalytic domains have been shown to influence substrate specificity in TACE and other ADAMs (25, 36).

The present study identifies significant differences between the inhibition of the long form of TACE and MMPs by TIMP-3. Firstly, the inhibition of TACE by wild-type N-TIMP-3 and two mutants displays positive cooperativity with Hill coefficients of 1.9 to 3.5. This observation was unexpected but has been confirmed with different preparations of TACE and also at a lower pH (7.5). Positive cooperativity arises from the presence of multiple interacting binding sites and alternative conformational states and its structural basis in TACE is currently unknown. However, positive cooperativity has been previously described for the hydrolysis of a synthetic peptide substrate by a similar form of TACE (37). Cooperativity was only observed with a peptide substrate derivatized at the N- and C-termini, whereas uncapped peptides showed normal hyperbolic saturation curves (37). This apparent allosteric behavior could have important implications for the regulation of TACE activity.

A second major difference in N-TIMP-3 inhibition is the observation that both the T2G and -1A mutants of N-TIMP-3 are potent inhibitors of TACE but are extremely weak inhibitors of the four representative MMPs (collagenase 1, gelatinase A, stromelysin 1 and membrane-type 1 MMP), and are likely also to be weak inhibitors of other MMPs. The presence of any extension N-terminal to the α-amino group in TIMPs, has been shown to drastically reduce inhibitory activity for MMPs (15-17), presumably because such extensions prevent the interaction of Cys1 with the catalytic Zn²⁺. The fact that the -1A mutant of N-TIMP-3 is an effective inhibitor of TACE but not MMPs suggests that the interaction of the inhibitor with the active site Zn²⁺ may be relatively unimportant for the strength of binding to TACE. This appears to be consistent with previous studies of TACE inhibition by its own pro-domain in which it was found that a bacterially-expressed form of the isolated pro-domain (residues 22 to 214) inhibits both the catalytic domain and the full-length soluble form of TACE. Mutation of CyS¹⁸⁴ of the cysteine switch region in the isolated pro-domain, which in MMPs interacts with the catalytic Zn²⁺ of the metalloproteinase domain, had no significant effect on pro-domain inhibition (26).

Another key feature of the interaction of TIMPs with MMPs is the extension of the side chain of residue 2 of the TIMPs into the S1′ specificity pocket of the MMPs. The corresponding residue has been proposed to have a similar role in the model of TIMP-3/TACE complex (34). As compared with most MMPs, the S1′ pocket of TACE is deep and very hydrophobic. However, substitution of Thr² of N-TIMP-3 by residues with larger hydrophobic side chains that should fit better into the S1′ site of TACE, failed to improve the binding of the inhibitor to this enzyme (21). Mutation of this residue into glycine, which lacks a side chain for potential interaction with the S1′ pocket of the protease, results in a major reduction in the affinity for MMPs, but has little effect on the inhibition of TACE. This suggests that this site of interaction also contributes little to the free energy of binding. We also cannot rule out the possibility that TIMP-3 is oriented in a different way in the complex with TACE than with MMPs, so that Thr² is not even in contact with the S1′ pocket of the enzyme.

The long form of TACE, used in the present work, differs from the catalytic domain in responses to inhibitors. It is more than 30-fold less sensitive to inhibition by the TACE pro-domain (26) and also more weakly inhibited by N-TIMP-3 (35). Furthermore, several mutations that enhance N-TIMP-3 binding to the TACE catalytic domain were found to have little effect on binding to the longer form of the enzyme (35). Murphy and co-workers have suggested that the cysteine-rich domain of TACE may act to inhibit TIMP-3 binding to the catalytic domain, and reported that mutation of lysines distant from the MMP reactive site produces inhibitors that are more effective with longer enzyme forms (22). These results suggest that the non-catalytic domains modulate the properties of the catalytic domain and emphasize the importance of considering the inhibitory properties of the longer enzyme forms in developing specific inhibitors for possible use in vivo.

Soluble TNF-α is released from cultured cells or tissues by several proteases besides TACE/ADAM17, including ADAM10, ADAM19, MMP-7 and the leucocyte serine protease, protease 3 (38-41). Although ADAM10, purified from the membrane extract of THP-1 cells, was shown to process pro-TNF-α in vitro (42), studies with antisense oligos specifically targeting different ADAM mRNAs suggest that TACE, but not ADAM10, is the major sheddase for TNF-α in this cell line (33). This agrees with our finding that N-TIMP-3 efficiently inhibits the shedding of TNF-α in THP-1 cells whereas the inhibitory domain of TIMP-1, a potent inhibitor of ADAM10, has no effect. The fact that N-TIMP-3 mutants that do not efficiently inhibit MMPs have similar effects to the wild-type inhibitor effectively rules out the possibility that MMPs make a major contribution to the shedding activity in these cells. These mutants provide useful tools for differentiating the activities of MMPs from that of TACE and possibly other ADAMs in biological systems. In the latter regard it is interesting to find out how these mutations affect the inhibitory activity of TIMP-3 for disintegrin-metalloproteinases.

The direct involvement of TIMP-3 in the inhibition of TNF-α shedding in vivo was demonstrated recently in a mouse model, where elimination of the TIMP-3 gene results in excessive TACE activity, elevated levels of soluble TNF-α and severe inflammation in the liver (43). This observation further validates the feasibility of using TIMP-3 in the therapy of inflammatory diseases that involve unregulated shedding of TNF-α including rheumatoid arthritis and Crohn's disease. However, although a series of MMPs are overexpressed in arthritis (44), the lack of MMP activities has been blamed for joint and bone abnormality. For example, MT1-MMP is indispensable for maintenance of a stable pool of osteocytes and normal development of bones (45), and mice with deficiency in the gene encoding MT1-MMP develop osteopenia and arthritis (46). Furthermore, two mutations in the MMP-2 gene, identified in a number of consanguineous Saudi Arabian families, result in loss of MMP-2 activity, and may be the cause of an autosomal recessive form of multicentric osteolysis and arthritis in affected family members (47). These observations suggest that MMPs may have important protective effects against arthritis. Since the N-terminal domain of TIMP-3 is a potent inhibitor of both MMP-2 and MT1-MMP (27), the outcome of the potential therapy using the wild-type inhibitor is unpredictable. The N-TIMP-3 mutants described here may have an advantage over the wild-type inhibitor in clinical applications, since they essentially spare the MMPs, a large family of proteases that have important roles in normal physiological processes.

### REFERENCES

1. Woessner, J. F., and Nagase, H. (2000) Matrix metalloproteinases and TIMPs. Oxford University Press, 126-127.
2. Moss, M. L., and Bartsch, J. G. (2004) Biochemistry 43, 7227-7235.
3. Blobel, C. P. (2005) Nat. Rev. Mol. Cell. Biol. 6, 32-43.
4. Nagase, H., and Brew, K. (2003) Biochem. Soc. Symp. 70, 201-212.
5. Amour, A., Knight, C. G., Webster, A., Slocombe, P. M., Stephens, P. E., Knauper, V., Docherty, A. J., and Murphy, G. (2000) FEBS Lett. 473, 275-279.
6. Loechel, F., Fox, J. W., Murphy, G., Albrechtsen, R., and Wewer, U. M. (2000) Biochem. Biophys. Res. Commun. 278, 511-515.
7. Amour, A., Slocombe, P. M., Webster, A., Butler, M., Knight, C. G., Smith, B. J., Stephens, P. E., Shelley, C., Hutton, M., Knauper, V., Docherty, A. J., and Murphy G. (1998) FEBS Lett. 435, 39-44.
8. Kashiwagi, M., Tortorella, M., Nagase, H., and Brew, K. (2001) J. Biol. Chem. 276, 12501-12504.
9. Brew, K., Dinakarpandian, D., and Nagase, H. (2000) Biochim. Biophys. Acta. 1477, 267-283.
10. Baker, A. H., Edwards, D. R., and Murphy, G. (2002) J Cell Sci. 115, 3719-3727.
11. Weber, B. H. F., Vogt, G., Pruett, R. C., Stohr, H. and Felbor, U. (1994) Nature Genet. 8, 352-356.
12.12. Qi, J. H., Ebrahem, Q., and Anand-Apte, B. (2003) Adv. Exp. Med. Biol. 533, 97-105.
13. Gomis-Ruth, F. X., Maskos, K., Betz, M., Bergner, A., Huber, R., Suzuki, K., Yoshida, N., Nagase, H., Brew, K., Bourenkov, G. P., Bartunik, H., and Bode, W. (1997) Nature 389, 77-81.
14. Fernandez-Catalan, C., Bode, W., Huber, R., Turk, D., Calvete, J. J., Lichte, A., Tschesche, H., and Maskos, K. (1998) EMBO J. 17, 5238-5248.
15. Higashi, S., and Miyazaki, K. (1999) J. Biol. Chem. 274, 10497-10504.
16. Troeberg, L., Tanaka, M., Wait, R., Shi, Y. E., Brew, K., and Nagase, H. (2002) Biochemistry 41, 15025-15035.
17. Wingfield, P. T., Sax, J. K., Stahl, S. J., Kaufman, J., Palmer, I., Chung, V., Corcoran, M. L., Kleiner, D. E., and Stetler-Stevenson, W. G. (1999) J. Biol. Chem. 274, 21362-21368.
18. Meng, Q., Malinovskii, V., Huang, W., Hu, Y., Chung, L., Nagase, H., Bode, W., Maskos, K., and Brew, K. (1999) J. Biol. Chem. 274, 10184-10189.
19. Wei, S., Chen, Y., Chung, L., Nagase, H., and Brew, K. (2003) J. Biol. Chem. 278, 9831-9834.
20. Maskos, K., Fernandez-Catalan, C., Huber, R., Bourenkov, G. P., Bartunik, H., Ellestad, G. A., Reddy, P., Wolfson, M. F., Rauch, C. T., Castner, B. J., Davis, R., Clarke, H. R., Petersen, M., Fitzner, J. N., Cerretti, D. P., March, C. J., Paxton, R. J., Black, R. A., and Bode, W. (1998) Proc. Natl. Acad. Sci. U.S.A. 95, 3408-3412.
21. Lee, M. H., Verma, V., Maskos, K., Nath, D., Knauper, V., Dodds, P., Amour, A., and Murphy, G. (2002) Biochem. J. 364, 227-234.
22. Lee, M. H., Dodds, P., Verma, V., Maskos, K., Knauper, V., and Murphy, G. (2003) Biochem. J., 371, 369-376.
23. Lee, M. H., Rapti, M., and Murphy, G. (2004) J. Biol. Chem. 279, 45121-45129.
24. Lee, M. H., Rapti, M., and Murphy, G. (2005) J. Biol. Chem. 280, 15967-15975.
25. Reddy, P., Slack, J. L., Davis, R., Cerretti, D. P., Kozlosky, C. J., Blanton, R. A., Shows, D., Peschon, J. J., and Black, R. A. (2000) J. Biol. Chem. 275, 14608-14614.
26. Gonzales, P. E., Solomon, A., Miller, A. B., Leesnitzer, M. A., Sagi, I., and Milla, M. E. (2004) J. Biol. Chem. 279, 31638-31645.
27. Wei, S., Xie, Z., Filenova, E., and Brew, K. (2003) Biochemistry 42, 12200-12207.
28. Milla, M. E., Leesnitzer, M. A., Moss, M. L., Clay, W. C., Carter, H. L., Miller, A. B., Su, J. L., Lambert, M. H., Willard, D. H., Sheeley, D. M., Kost, T. A., Burkhart, W., Moyer, M., Blackburn, R. K., Pahel, G. L., Mitchell, J. L., Hoffman, C. R., and Becherer, J. D. (1999) J. Biol. Chem. 274, 30563-30570.
29. Cortez, A., Cascante, M., Cardenas, M. L., and Cornish-Bowden, A. (2001) Biochem. J. 357, 263-268.
30. Engelberts, I., Moller, A., Schoen, G. J., van der Linden, C. J., and Buurman, W. A. (1991) Lymphokine Cytokine Res. 10, 69-76.
31. Lee, M. H., Rapti, M., and Murphy, G. (2003) J. Biol. Chem. 278, 40224-40230.
32. Black, R. A., Rauch, C. T., Kozlosky, C. J., Peschon, J. J., Slack, J. L., Wolfson, M. F., Castner, B. J., Stocking, K. L., Reddy, P., Srinivasan, S., Nelson, N., Boiani, N., Schooley, K. A., Gerhart, M., Davis, R., Fitzner, J. N., Johnson, R. S., Paxton, R. J., March, C. J., and Cerretti, D. P. (1997) Nature 385, 729-733.
33. Condon, T. P., Flournoy, S., Sawyer, G. J., Baker, B. F., Kishimoto, T. K., and Bennett, C. F. (2001) Antisense Nucleic Acid Drug Dev. 11, 107-116.
34. Lee, M. H., Maskos, K., Knauper, V., Dodds, P., and Murphy, G. (2002) Protein Sci. 11, 2493-2503.
35. Lee, M. H., Verma, V., Maskos, K., Becherer, J. D., Knauper, V., Dodds, P., Amour, A., and Murphy, G. (2002) FEBS Lett. 520, 102-106.
36. Smith, K. M., Gaultier, A., Cousin, H., Alfandari, D., White, J. M., and DeSimone, D. W. (2002) J. Cell Biol. 159, 893-902.
37. Jin, G., Huang, X., Black, R., Wolfson, M., Rauch, C., McGregor, H., Ellestad, G., and Cowling, R. (2002) Anal. Biochem. 302, 269-275.
38. Lunn, C. A., Fan, X., Dalie, B., Miller, K., Zavodny, P. J., Narula, S. K., and Lundell, D. (1997) FEBS Lett. 400, 333-335.
39. Zheng, Y., Saftig, P., Hartmann, D., and Blobel, C. (2004) J. Biol. Chem. 279, 42898-42906.
40. Haro, H., Crawford, H. C., Fingleton, B., Shinomiya, K., Spengler, D. M., and Matrisian, L. M. (2000) J. Clin. Invest. 105, 143-150.
41. Coeshott, C., Ohnemus, C., Pilyavskaya, A., Ross, S., Wieczorek, M., Kroona, H., Leimer, A. H., and Cheronis, J. (1999) Proc. Natl. Acad. Sci. U.S.A. 96, 6261-6266.
42. Rosendahl, M. S., Ko, S. C., Long, D. L., Brewer, M. T., Rosenzweig, B., Hedi, E., Anderson, L., Pyle, S. M., Moreland, J., Meyers, M. A., Kohno, T., Lyons, D., and Lichenstein, H. S. (1997) J. Biol. Chem. 272, 24588-24593.
43. Mohammed, F. F., Smookler, D. S., Taylor, S. E., Fingleton, B., Kassiri, Z., Sanchez, O. H., English, J. L., Matrisian, L. M., Au, B., Yeh, W. C., and Khokha, R. (2004) Nat. Genet. 36, 969-977.
44. Martel-Pelletier, J., Welsch, D. J., and Pelletier, J. P. (2001) Best Pract. Res. Clin. Rheumatol. 15, 805-829.
45. Holmbeck, K., Bianco, P., Pidoux, I., Inoue, S., Billinghurst, R. C., Wu, W., Chrysovergis, K., Yamada, S., Birkedal-Hansen, H., and Poole, A. R. (2005) J Cell Sci. 118, 147-156.
46. Holmbeck, K., Bianco, P., Caterina, J., Yamada, S., Kromer, M., Kuznetsov, S. A., Mankani, M., Robey, P. G., Poole, A. R., Pidoux, I., Ward, J. M., and Birkedal-Hansen, H. (1999) Cell 99, 81-92.
47. Martignetti, J. A., Aqeel, A. A., Sewairi, W. A., Boumah, C. E., Kambouris, M., Mayouf, S. A., Sheth, K. V., Eid, W. A., Dowling, O., Harris, J., Glucksman, M. J., Bahabri, S., Meyer, B. F., and Desnick, R. J. (2001) Nature. Genet. 28, 261-265.
48. Kopp, J., and Schwede, T. (2004) Nucleic Acids Res. 32, D230-D234.
49. Pettersen, E. F., Goddard, T. D., Huang, C. C., Couch, G. S., Greenblatt, D. M., Meng, E. C., and Ferrin, T. E. (2004) J. Comput. Chem. 25, 1605-1612.

TABLE I
Kᵢ^{(app)} (nM) of wild-type and mutant N-TIMP-3 with some MMPs.

| | MMP-1 | MMP-2 | MMP-3(ΔC) | MMP-14(CD) |
|---|---|---|---|---|
| WT | 1.2 ± 0.5* | 4.3 ± 0.5* | 67 ± 2.8* | 0.8 ± 0.03** |
| T2G | 547 ± 100 | ~4.2 × 10³ | >1 × 10⁴ | ~2.3 × 10³ |
| -1A | ~1.3 × 10³ | 614 ± 32 | ~3.3 × 10³ | 941 ± 206 |

Concentration of enzymes used: MMP-1 and MMP-14(CD), 5 nM; MMP-2 and MMP-3(AC), 1 nM.
*Data taken from ref. 8.
**Data taken from ref. 27.

TABLE II
Comparison of inhibition parameters for TACE
of N-TIMP-3 and its mutants with TAPI-2.

| | Kᵢ^{(app)} (nM) | h |
|---|---|---|
| WT | 13.7 ± 0.2 | 3.59 ± 0.16 |
| T2G | 35.6 ± 1.9 | 2.54 ± 0.25 |
| -1A | 33.9 ± 2.8 | 1.9 ± 0.22 |
| TAPI-2 | 4.28 ± 0.001 | 1 |

Kᵢ^{(app)} and h values were calculated by fitting the data from FIG. 2 with equation 3.

### EXAMPLE 2

### Test of (-1A)N-TIMP-3 and N-TIMP-3(T2G) Mutants for Their Ability to Block ADAMTS-4

### Methods:

Recombinant human ADAMTS-4 lacking the C-terminal spacer domain was prepared and expressed as described (Kashimagi, M. et al. J. Biol. Chem. 279, 10109-10119, 2004), and bovine cartilage aggrecan was purified according to Hascall and Sajdesa (J. Biol. Chem. 244, 2384-2396, 1969). Antibody that recognized the fragment with the C-terminal GELE was described by Kashiwagi et al (2004). To examine the inhibition of ADAMTS-4 by (-1A) N-TIMP-3 and N-TIMP-3(T2G), 0.5 nM ADAMTS-4 was incubated with a various concentration of the inhibitor for 30 mins at room temperature and then with 1 mg/ml of bovine aggrecan at 37° C. for 2 h. The reaction was stopped by 10 mM EDTA, and the digestion products were deglycosylated by chondroitinase ABC (0.01 unit/10 g of aggrecan) and keratanase (0.01 unit/10 μg of aggrecan) in Tris-acetate (pH 6.5), 5 mM EDTA at 37° C. for 3 h. The products were then precipated with 10 vol. of acetone and subjected to Western blotting analysis with the anti-GELE antibody as primary antibody and developed as described by Little et al. The staining intensity of the band was quantified by densitometric analysis.

### Results:

Both (-1A) N-TIMP-3 (left panel) and N-TIMP-3(T2G) (right panel) show dose-dependent inhibition with the Kᵢ^{(app)} of 18 nM and 15 nM, respectively.

### Discussion:

In vitro inhibition assay indicates that N-TIMP-3 mutants are effective inhibitors of ADAMTS-4 (aggrecanase 1). Because N-TIMP-3 inhibits both ADAMTS4 and ADAMTS-5 (aggrecanase 2) (Kashwagi et al, 2001 [147]), we postulate that these mutants are likely to inhibit ADAMTS-5 to a similar extent. Therefore these N-TIMP-3 mutants are likely to be effective inhibitors of cartilage aggrecan degradation.

### REFERENCES

Kashiwagi, M., Tortorella, M., Nagase, H. and Brew, K. (2001) J Biol Chem 276, 12501-4.
Little, C. B., Flannery, C. R., Hughes, C. E., Mort, J. S., Roughley, P. J., Dent, C. and Caterson, B. (1999) Biochem J 344, 61-8.
Kashiwagi et al 2004, JBC 279, 10109-10119

### EXAMPLE 3

### Test of (-1A)N-TIMP-3 and N-TIMP-3(T2G) Mutants for Their Ability to Block Cartilage Aggrecan Degradation Using Porcine Articular Cartilage in Culture

### Cartilage Culture and Inhibition Studies

Porcine articular cartilage from the metacarpophalangeal joints of 3-9 month old pigs is dissected into small shavings approximately 3 mm long and 2-3 mm wide. After dissection, the cartilage is allowed to rest for 24 h at 37° C. under 5% CO₂ in DMEM containing penicillin-streptomycin, amphotericin B, and 5% fetal calf serum. The medium is then replaced with fresh media and the cartilage is rested for a further 24-48 h. Each cartilage piece is then placed in one well of a round bottom 96-well plate with 200 μl of serum-free DMEM with or without 10-100 ng/ml IL-1α or 1 μM retinoic acid and various concentrations of each TIMP-3 mutant. After 3 days, all of the conditioned media are harvested and stored at −20° C. until use.

### Analysis of Glycosaminoglycan (GAG) Release

GAG released into the conditioned media is measured in duplicate using a modification of the dimethylmethylene blue (DMMB) assay as described in Farndale et al. [20]. Shark chondroitin sulfate (0-2.62 μg) is used as standard. The % of total GAG released into the medium is calculated as follows: % of total GAG released=(total GAG in the medium)/(total GAG in the medium+total GAG remaining in the cartilage).

### Identification of Aggrecanase- and MMP-Generated Aggrecan Fragments by Western Analysis

Aggrecan fragments released into the conditioned medium are deglycosylated by digestion with chondroitinase ABC and keratanase and the samples are subjected to SDS/PAGE and Western blotting analysis as described by Little et al. [17]. The primary antibodies used to detect aggrecanase-generated and MMP-generated aggrecan fragments are BC-3 and BC-14, respectively [19]. Antigen-antibody complexes are detected by anti-mouse AP-linked donkey antibody and the AP substrate.

### Results

The results of performing the above experiments using N-TIMP-3 are as follows. See also Gendron et al (2003) FEBS Lett 27877, 1-6. Similar results are considered likely with (-1A)N-TIMP-3) and N-TIMP-3(T2G) mutants.

### N-TIMP-3 Inhibits IL-1α- and Retinoic Acid-Stimulated Aggrecan Breakdown in Cartilage Explants

Bovine nasal cartilage explants were stimulated with IL-1α in the presence or absence of N-TIMP-1, TIMP-2, or N-TIMP-3 for 3 days. Explants treated with IL-1α showed approximately a 5-fold increase in GAG release over controls. The IL-1α-stimulated release was significantly inhibited by the addition of N-TIMP-3 in a concentration dependant manner. However, N-TIMP-1 and TIMP-2 were not effective even at the concentration of 1 μM. Safranin O staining of the cartilage explants upon treatment with IL-1α revealed that the addition of N-TIMP-3 did protect against the release of GAGs from the matrix. Similar results were observed with IL-1α-stimulated porcine articular cartilage.

The GAG release from porcine articular cartilage stimulated with retinoic acid was also inhibited by N-TIMP-3, but to a lesser extent compared with the IL-1α-stimulated cartilage. N-TIMP-1 and TIMP-2 did not inhibit the retinoic acid-stimulated GAG release.

### Aggrecanase Activity is Specifically Inhibited by N-TIMP-3

Conditioned media from the above experiments were analyzed by monoclonal antibodies that recognize either the aggrecanase-generated aggrecan neoepitope ARGSV or the MMP-generated aggrecan neoepitope FFGVG. In concordance with GAG release, there was an increase in the amount of aggrecanase-generated aggrecan fragments released upon treatment with either stimulus, but no MMP-generated fragments were detected. The release of aggrecanase-generated fragments was partially inhibited by 0.05 μM N-TIMP-3 and completely blocked by 0.1 μM N-TIMP-3 in both IL-1α- and retinoic acid-stimulated cartilage. N-TIMP-1 and TIMP-2 were not effective even at the concentration of 1 μM.

### Inhibition of IL-1α Stimulated Porcine Articular Cartilage Degradation by N-Terminal Mutants of N-TIMP-3

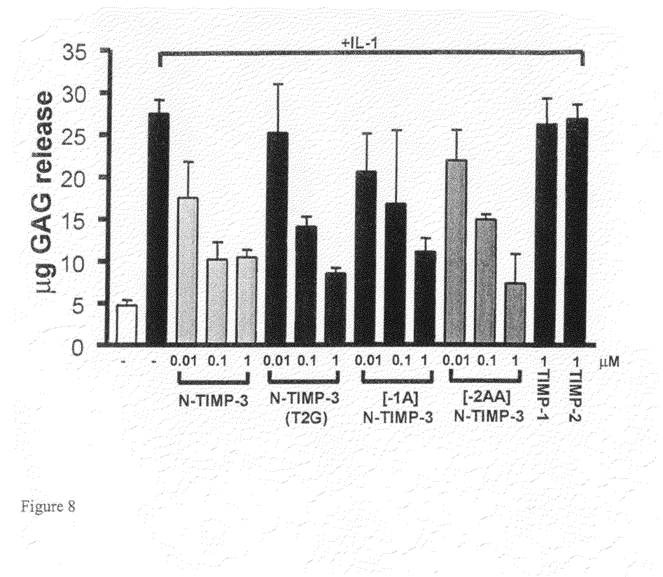

Porcine articular cartilage pieces were cultured for 3 days. Cartilage was stimulated with IL-1α (10 ng/ml) with TIMPs at the concentrations indicated. Glycosaminoglycan (GAG) release in the media was measured by dimethyl methylene blue (DMMB). N-TIMP-3 and the N-terminal mutants dose dependently inhibited degradation whereas TIMP-1 and TIMP-2 did not (FIG. 8).

### REFERENCES

### Numbering for Example 3

[17] Little, C. B., Flannery, C. R., Hughes, C. E., Mort, J. S., Roughley, P. J., Dent, C. and Caterson, B. (1999) Biochem J 344, 61-8.
[19] Hughes, C. E., Caterson, B., Fosang, A. J., Roughley, P. J. and Mort, J. S. (1995) Biochem J 305, 799-804.
[20] Farndale, R. W., Buttle, David J., and Barrett, Alan J. (1986) Biochimica et Biophysica Acta 883, 173-177.

### EXAMPLE 4

### Kᵢ₍ₐₚₚ₎ Determinations

### Assay for Aggrecanase (ADAMTS-4 and ADAMTS-5) Activity

### 1) Preparation of the GST-IGD-FLAG Substrate

The substrate containing glutathione S-transferase (GST) fused with the interglobular domain (IGD) of aggrecan (Tyr³³¹ to Gly⁴⁵⁷) attached with a C-terminal FLAG sequence (GST-IGD-FLAG) was prepared by cloning it into pGEX-4T1 at the EcoR1 and Xho1 cloning sites. This substrate was expressed in E. coli strain BL-21 (non-DE3) transfected with the pGEX4T1 GST-IGD-FLAG plasmid by induction with 100 mM isopropyl-beta-D-thiogalactopyranoside (IPTG). After induction, bacteria were collected by centrifugation and resuspended in 20 ml of 50 mM Tris-HCl (pH 8.0), 150 mM NaCl, 0.02% NaN₃, 100 mM DTT, 100 mM EDTA with proteinase inhibitor cocktail set II inhibitors (Merck, Nottingham, UK). The resuspended bacteria were then disrupted mechanically using a French Press (5×1500 Psi). After centrifugation at 24,000 g (30 min, 4° C.), the supernatant, containing the expressed GST-IGD-FLAG, was applied to a glutathione-Sepharose 4B column (Qiagen, Crawley, UK). The column was washed with 0.5 M NaCl, 50 mM Tris-HCl (pH 8.0) and eluted with 10 mM reduced glutathione, 50 mM Tris-HCl (pH 8.0). The eluted material was dialysed three times against 10 volumes of 50 mM Tris-HCl (pH 8.0), 150 mM NaCl. This substrate is then concentrated if necessary to A₂₈₀>2.5 using polyethyl sulphate membrane spin concentrators (Vivascience, Epsom, UK). The concentration of intact substrate (52 kDa) was determined by comparison with Coomassie Brilliant Blue staining of known amounts of bovine serum albumin (GE Healthsciences, Buckinghampshire, UK). The yield of substrate (>20 mg of partially purified material) per litre of bacterial culture was sufficient for over 2000 assay reactions.

### 2) Aggrecanase Assays

Aggrecanase assays were carried out in 50 mM Tris HCl pH 7.5, 150 mM NaCl, 10 mM CaCl₂, 0.02% NaN₃, 0.05% Brij-35 at 37° C. When N-TIMP-3 was used, the inhibitor was preincubated with the enzyme for 1 hour. Reaction volumes were a total of 10 μl, consisting of 5 μl of GST-IGD-FLAG substrate (34 μM), and 5 μl of ADAMTS-4 or ADAMTS-5 (2 nM) with or without inhibitor. Enzyme amounts and incubation times were as indicated. Enzyme reactions were stopped at appropriate time points with the addition of 10 μl 2×SDS-PAGE samples loading buffer containing 20 mM EDTA. Reactions were then applied to a 10% SDS-PAGE analysis. Proteins were stained using Coomassie Brilliant Blue R-250. The stained gels were then scanned using a scanning densitometer (Biorad GS-710, Hemel Hempstead, UK) and the band intensity of the product (17 kDa) quantified using the 1D Phoretix quantification software (Nonlinear Dynamics, Newcastle upon Tyne, UK). Background subtraction was done using the rolling ball method, and band intensity expressed as pixel volumes.

Kᵢ₍ₐₚₚ₎ determinations for MMP-1, MMP-2 and MMP-3 were performed as set out in Example 1.

TABLE 3
Summary of the Kᵢ₍ₐₚₚ₎ data of the N-terminal reactive site mutants
against MMP-1, MMP-2, MMP-3, ADAMTS-4, ADAMTS-5.

| | Kᵢ₍ₐₚₚ₎ | | | | |
|---|---|---|---|---|---|
| | (nM) | | | | |
| | MMP-1 | MMP-2 | MMP-3 | ADAMTS-4 | ADAMTS-5 |
| N-TIMP-3 | 8.2 ± 2 | 5.1 ± 1 | 1.8 ± 0.4 | 2.4 ± 1.6 | 0.4 ± 0.2 |
| N-TIMP-3 | >500 | >1000 | >1000 | 20 ± 3 | 1.5 ± 0.3 |
| T2G | | | | | |
| (-1A) N- | >500 | >1000 | >1000 | 25 ± 3 | 1.9 ± 0.6 |
| TIMP-3 | | | | | |
| (-2A) N- | >500 | >250 | >250 | 67 ± 30 | 1.4 ± 0.6 |
| TIMP-3 | | | | | |

The mutant (-2A)N-TIMP-3 inhibits ADAMTS-5 about 45 times more potently than ADAMTS-4. Recent studies using ADAMTS-4 and ADAMTS-5 null mice indicated that ADAMTS-5 is a key aggrecanase that causes cartilage destruction in a rheumatoid arthritis animal model (Stanton et al., 2005) and in an osteoarthritis animal model (Glasson et al., 2005). Our studies shown in FIG. 8 indicate that the three N-TIMP-3 mutants were as effective as the wild-type N-TIMP-3, suggesting also that the key aggrecanase is ADAMTS-5. Furthermore, our studies indicate that the (-2A)N-TIMP-3 mutant will be less toxic as it is more selective for ADAMTS-5.

(-2A)N-TIMP-3 is also a potent inhibitor of TACE. About 80-90% inhibition of TACE activity was observed with 100 nM (-2A)N-TIMP-3 whereas no inhibition was observed for MMP-1, -2 or -3 at this concentration.

### REFERENCES

Glasson, S. S., Askew, R., Sheppard, B., Carito, B., Blanchet, T., Ma, H. L., Flannery, C. R., Peluso, D., Kanki, K., Yang, Z., et al. (2005). Deletion of active ADAMTS5 prevents cartilage degradation in a murine model of osteoarthritis. Nature 434, 644-648.
Stanton, H., Rogerson, F. M., East, C. J., Golub, S. B., Lawlor, K. E., Meeker, C. T., Little, C. B., Last, K., Farmer, P. J., Campbell, I. K., et al. (2005). ADAMTS5 is the major aggrecanase in mouse cartilage in vivo and in vitro. Nature 434, 648-652.

### EXAMPLE 5

### Effect of TIMP-3 Mutants on Monocyte-Derived Macrophages (MDM)

MDMs release pro-inflammatory cytokines, including interleukin (IL)-1β, tumour necrosis factor (TNF)α and IL-6; chemokines, including IL-8 and matrix metalloproteases (MMP)-2, and -9, for example after stimulation with LPS. MDMs are therefore suitable cells on which to test the effects of the TIMP-3 mutants or compounds that are expected to inhibit an ADAM metalloproteinase to a greater extent than an MMP, as discussed above.

### Experimental Model

To address the efficacy of TMP-3 mutants on MDM, MDM from a healthy subject were cultured in the laboratory and stimulated with LPS. The effect of TIMP-3 mutants on MMP-9 activity and TNFα was measured.

### Methods

Isolation of Leukocytes from Peripheral Human Blood.

This method was adapted from Dransfield et al., [7] and performed under sterile conditions. Blood was collected into EDTA (2% W/v). Dextran solution (6% w/v) was added to the whole blood in volumes of 10 ml per 20 ml blood and the final volume adjusted to 50 ml in Dulbecco's PBS. The samples were allowed to sediment at room temperature for 45 min. After sedimentation, the upper leukocyte rich layer was centrifuged at 400×g for 10 min, 4° C. and the supernatant discarded. The pellets containing the cells were resuspended in Dulbecco's PBS and centrifuged for a second time as before.

### Gradient Preparation

The gradient consisted of three separate concentrations of Percoll™. A ‘100% v/v’ Percoll™ solution was prepared from 90% v/v Percoll™ containing 10% v/v 10×PBS. The gradient was then prepared as follows: 4 ml 81% v/v Percoll was added to a 15 ml Falcon tube. This was overlaid by 4 ml 70% v/v Percoll. The cell pellet was resuspended in 3 ml of 55% v/v Percoll™ and then overlaid onto the pre-prepared gradient. The cells were centrifuged at 750×g for 20 min at 4° C. The peripheral blood mononuclear cells (PBMCs) were harvested from the 55%/70% interface (top layer) and the polymorphonuclear (PMN) cells remained at the 70%/81% interface (bottom layer). PBMCs were washed twice by centrifugation in sterile PBS.

### Monocyte Isolation Using a VarioMACS and Negative Selection Magnetic Labelling.

PBMCs were washed in serum-containing separation buffer (sterile PBS, 0.5% w/v bovine serum albumin (BSA), 2 mM EDTA. The cells were diluted 1:100 in Kimura stain and counted using a haemocytometer. The cell pellet was resuspended with the following ratio of reagents from the monocyte isolation kit. 60 μl separation buffer, 20 μl Fc receptor (FcR) blocking reagent and 20 μl Hapten Conjugated Antibody cocktail were added to 10⁷ cells and incubated at 6-12° C. for 5 min. The cells were washed twice (5 min, 4° C., 250×g) in separation buffer in a volume 10-20 times higher than the labelling volume. The cell pellet was resuspended in: 60 μl separation buffer, 20 μl FcR blocking reagent, 20 μl MACS anti-hapten microbeads and 5 μl CD15 microbeads (to remove any contaminating neutrophils) per 10⁷ cells and incubated at 6-12° C. for 15 min. The cells were washed (5 min, 4° C., 250×g) and resuspended in 500 μl separation buffer. The magnetic column was prepared by washing with 3 ml separation buffer. The cell suspension was added and the column washed a further 4 times with 3 ml aliquots of separation buffer. The magnetic column filtrate containing the monocytes was washed twice in MDM medium (RPMI 1640 containing phenol red, 10% v/v heat inactivated FBS (HIFBS), 10,000 u/10 mg/ml (1% v/v) penicillin/streptomycin, 2 mM (1% v/v) L-Glutamine).

### Cell Culture Techniques for Monocyte-Derived Macrophages.

Monocytes were seeded at a density of 1×10⁵ cells/well in a 96-well tissue culture treated Costar™ plate and cultured for 12 d at 37° C. at 5% v/v CO₂ in a humidified incubator. The medium and 2 ng/ml GM-CSF were changed on day 4 and 8. On day 12, cells had differentiated into the macrophage phenotype.

### Assays

TNFα was measured using a commercially available ELISA kit and MMP-9 was measured using a Fluorokine kit.

### Results

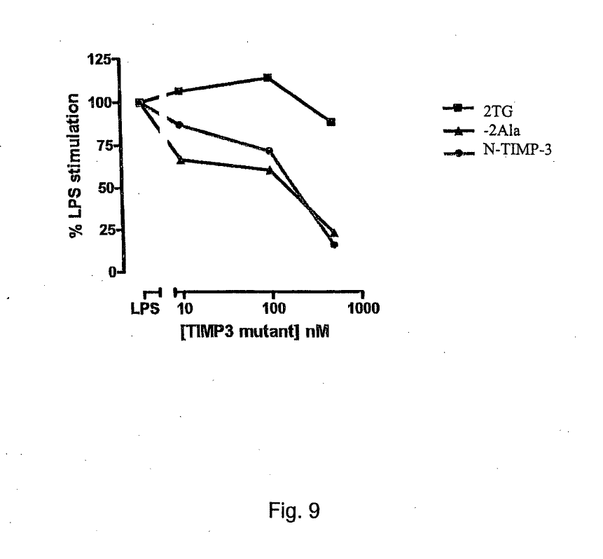

The N-TIMP-3 mutant T2G had little effect on basal TNFα release by MDM. In the presence of LPS, T2G had little effect on inhibition of LPS stimulated TNFα release by MDM (FIG. 9).

The N-TIMP-3 mutant -2Ala had little effect on basal TNFα release by MDM. However, in the presence of LPS, -2Ala mutant inhibited LPS stimulated TNFα release by MDM with an EC₅₀ of ˜180 nM (FIG. 9).

Similar to the -2Ala TIMP-3 mutant, the N-TIMP-3 molecule also had little effect on basal TNFα release by MDM. Again, this polypeptide inhibited LPS stimulated TNFα release by MDM with an EC₅₀ of ˜180 nM (FIG. 9).

The effect of these mutants on cells from a normal subject indicate that these mutants can be of benefit to reduce TNFα levels.

1. A mutant TIMP-3 (Tissue Inhibitor of MetalloProteinase-3) polypeptide wherein an additional residue, or 1 up to 2, 3, 4, 5, 6, 8, 10, 12, 15, 18 or 20 residues, lies immediately on the amino-terminal side of the first amino acid residue (Cys1) of the mature TIMP-3 polypeptide; or wherein the residue corresponding to Threonine2 of TIMP-3 is mutated to Glycine, or another of the following L-amino acids: Ala, Cys, Asp, Glu, Phe, His, Ile, Lys, Asn, Pro, Gln, Arg, Val, Trp.

2-14. (canceled)

15. The mutant TIMP-3 polypeptide of claim 1 wherein the additional amino acid residue (or further residue or residues) on the amino-terminal side of the first amino acid residue (Cys1) of the TIMP-3 polypeptide is selected from the group consisting of an L-Alanine residue, Gly and one of the following L-amino acids: Asp, Cys, Glu, Phe, His, Ile, Lys, Leu, Met, Asn, Pro, Gln, Arg, Ser, Thr, Val, Trp, Tyr.

16. The mutant TIMP-3 polypeptide of claim 1 wherein the mutant TIMP-3 polypeptide has or comprises the amino acid sequence selected from the group consisting of
xctcspshpqdafcnsdivirakvvgkklvkegpfgtlvytikqmkmyr
gftkmphvqyihteaseslcglklevnkyqylltgrvydgkmytglcnf
verwdqltlsqrkglnyryhlgcnckikscyylpcfvtskneclwtdml
snfgypgyqskhyacirqkggycswyrgwappdksiinatdp
wherein x is selected from the group consisting of a or one of the following: d, e, f, g, h, i, k, l, m, n, p, q, r, s, t, v, w, y
or the sequence
aactcspshpqdafcnsdivirakvvgkklvkegpfgtlvytikqmkmy
rgftkmphvqyihteaseslcglklevnkyqylltgrvydgkmytglcn
fverwdqltlsqrkglnyryhlgcnckikscyylpcfvtskneclwtdml
snfgypgyqskhyacirqkggycswyrgwappdksiinatdp
or the sequence
czcspshpqdafcnsdivirakvvgkklvkegpfgtlvytikqmkmyr
gftkmphvqyihteaseslcglklevnkyqylltgrvydgkmytglcnf
verwdqltlsqrkglnyryhlgcnckikscyylpcfvtskneclwtdmls
nfgypgyqskhyacirqkggycswyrgwappdksiinatdp
wherein z is selected from the group consisting of g or one of the following: a, d, e, f, h, i, k, n, p, q, r, v, w
or the sequence
xczcspshpqdafcnsdivirakvvgkklvkegpfgtlvytikqmkmyrg
ftkmphvqyihteaseslcglklevnkyqylltgrvydgkmytglcnfve
rwdqltlsqrkglnyryhlgcnckikscyylpcfvtskneclwtdmlsnf
gypgyqskhyacirqkggycswyrgwappdksiinatdp
wherein x is selected from the group consisting of a or one of the following: d, e, f, g, h, i, k, l, m, n, p, q, r, s, t, v, w, y and z is selected from the group consisting of g or one of the following: a, d, e, f, h, i, k, n, p, q, r, v, w
or the sequence
xctcspshpqdafcnsdivirakvvgkklvkegpfgtlvytikqmkmyrg
ftkmphvqyihteaseslcglklevnkyqylltgrvydgkmytglcnfve
rwdqltlsqrkglnyryhlgcn
wherein x is selected from the group consisting of a or one of the following: d, e, f, g, h, i, k, l, m, n, p, q, r, s, t, v, w, y
or the sequence
aactcspshpqdafcnsdivirakvvgkklvkegpfgtlvytikqmkmyr
gftkmphvqyihteaseslcglklevnkyqylltgrvydgkmytglcnfv
erwdqltlsqrkglnyryhlgcn
or the sequence
czcspshpqdafcnsdivirakvvgkklvkegpfgtlvytikqmkmyrgf
tkmphvqyihteaseslcglklevnkyqylltgrvydgkmytglcnfver
wdqltlsqrkglnyryhlgcn
wherein z is selected from the group consisting of g or one of the following: a, d, e, f, h, i, k, n, p, q, r, v, w
or the sequence
xczcspshpqdafcnsdivirakvvgkklvkegpfgtlvytikqmkmyrg
ftkmphvqyihteaseslcglklevnkyqylltgrvydgkmytglcnfve
rwdqltlsqrkglnyryhlgcn
wherein x is selected from the group consisting of a or one of the following: d, e, f, g, h, i, k, l, m, n, p, q, r, s, t, v, W, y and z is selected from the group consisting of g or one of the following: a, d, e, f, h, i, k, n, p, q, r, v, w.

17. The mutant TIMP-3 polypeptide of claim 15 wherein the mutant TIMP-3 polypeptide has or comprises the amino acid sequence selected from the group consisting of
xctcspshpqdafcnsdivirakvvgkklvkegpfgtlvytikqmkmyrg
ftkmphvqyihteaseslcglklevnkyqylltgrvydgkmytglcnfve
rwdqltlsqrkglnyryhlgcnckikscyylpcfvtskneclwtdmlsnf
gypgyqskhyacirqkggycswyrgwappdksiinatdp
wherein x is selected from the group consisting of a or one of the following: d, e, f, g, h, i, k, l, m, n, p, q, r, s, t, v, w, y
or the sequence
aactcspshpqdafcnsdivirakvvgkklvkegpfgtlvytikqmkmyr
gftkmphvqyihteaseslcglklevnkyqylltgrvydgkmytglcnfv
erwdqltlsqrkglnyryhlgcnckikscyylpcfvtskneclwtdmlsn
fgypgyqskhyacirqkggycswyrgwappdksiinatdp
or the sequence
czcspshpqdafcnsdivirakvvgkklvkegpfgtlvytikqmkmyrgf
tkmphvqyihteaseslcglklevnkyqylltgrvydgkmytglcnfver
wdqltlsqrkglnyryhlgcnckikscyylpcfvtskneclwtdmlsnfg
ypgyqskhyacirqkggycswyrgwappdksiinatdp
wherein z is selected from the group consisting of g or one of the following: a, d, e, f, h, i, k, n, p, q, r, v, w
or the sequence
xczcspshpqdafcnsdivirakvvgkklvkegpfgtlvytikqmkmyrg
ftkmphvqyihteaseslcglklevnkyqylltgrvydgkmytglcnfve
rwdqltlsqrkglnyryhlgcnckikscyylpcfvtskneclwtdmlsnf
gypgyqskhyacirqkggycswyrgwappdksiinatdp
wherein x is selected from the group consisting of a or one of the following: d, e, f, g, h, i, k, l, m, n, p, q, r, s, t, v, w, y and z is selected from the group consisting of g or one of the following: a, d, e, f, h, i, k, n, p, q, r, v, w
or the sequence
xctcspshpqdafcnsdivirakvvgkklvkegpfgtlvytikqmkmyrg
ftkmphvqyihteaseslcglklevnkyqylltgrvydgkmytglcnfve
rwdqltlsqrkglnyryhlgcn
wherein x is selected from the group consisting of a or one of the following: d, e, f, g, h, i, k, l, m, n, p, q, r, s, t, V, w, y
or the sequence
aactcspshpqdafcnsdivirakvvgkklvkegpfgtlvytikqmkmyr
gftkmphvqyihteaseslcglklevnkyqylltgrvydgkmytglcnfv
erwdqltlsqrkglnyryhlgcn
or the sequence
czcspshpqdafcnsdivirakvvgkklvkegpfgtlvytikqmkmyrgf
tkmphvqyihteaseslcglklevnkyqylltgrvydgkmytglcnfver
wdqltlsqrkglnyryhlgcn
wherein z is selected from the group consisting of g or one of the following: a, d, e, f, h, i, k, n, p, q, r, v, w
or the sequence
xczcspshpqdafcnsdivirakvvgkklvkegpfgtlvytikqmkmyrg
ftkmphvqyihteaseslcglklevnkyqylltgrvydgkmytglcnfve
rwdqltlsqrkglnyryhlgcn
wherein x is selected from the group consisting of a or one of the following: d, e, f, g, h, i, k, l, m, n, p, q, r, s, t, v, w, y and z is selected from the group consisting of g or one of the following: a, d, e, f, h, i, k, n, p, q, r, v, w.

18. A polynucleotide encoding a mutant TIMP-3 polypeptide according to claim 1.

19. A polynucleotide as in claim 16 comprising a polynucleotide sequence selected from the group consisting of
gcxtgcacatgctcgcccagccacccccaggacgccttctgcaactccga
catcgtgatccgggccaaggtggtggggaagaagctggtaaaggaggggc
ccttcggcacgctggtctacaccatcaagcagatgaagatgtaccgaggc
ttcaccaagatgccccatgtgcagtacatccacacggaagcttccgagag
tctctgtggccttaagctggaggtcaacaagtaccagtacctgctgacag
gtcgcgtctatgatggcaagatgtacacggggctgtgcaacttcgtggag
aggtgggaccagctcaccctctcccagcgcaaggggctgaactatcgg
tatcacctgggttgtaactgcaagatcaagtcctgctactacctgccttg
ctttgtgacttccaagaacgagtgtctctggaccgacatgctctccaatt
tcggttaccctggctaccagtccaaacactacgcctgcatccggcaga
agggcggctactgcagctggtaccgaggatgggcccccccggataaaagc
atcatcaatgccacagacccc
where x can be t, c, a or g,
or
gcxgcxtgcacatgctcgcccagccacccccaggacgccttctgcaactc
cgacatcgtgatccgggccaaggtggtggggaagaagctggtaaaggagg
ggcccttcggcacgctggtctacaccatcaagcagatgaagatgtaccga
ggcttcaccaagatgccccatgtgcagtacatccacacggaagcttccga
gagtctctgtggccttaagctggaggtcaacaagtaccagtacctgctga
caggtcgcgtctatgatggcaagatgtacacggggctgtgcaacttcgtg
gagaggtgggaccagctcaccctctcccagcgcaaggggctgaactatcg
gtatcacctgggttgtaactgcaagatcaagtcctgctactacctgcct
tgctttgtgacttccaagaacgagtgtctctggaccgacatgctctccaa
tttcggttaccctggctaccagtccaaacactacgcctgcatccggcaga
agggcggctactgcagctggtaccgaggatgggcccccccggataaaa
gcatcatcaatgccacagacccc
where x can be t, C, a or g,
or
tgcggxtgctcgcccagccacccccaggacgccttctgcaactccgacat
cgtgatccgggccaaggtggtggggaagaagctggtaaaggaggggccct
tcggcacgctggtctacaccatcaagcagatgaagatgtaccgaggcttc
accaagatgccccatgtgcagtacatccacacggaagcttccgagagtct
ctgtggccttaagctggaggtcaacaagtaccagtacctgctgacaggtc
gcgtctatgatggcaagatgtacacggggctgtgcaacttcgtggagagg
tgggaccagctcaccctctcccagcgcaaggggctgaactatcggtatca
cctgggttgtaactgcaagatcaagtcctgctactacctgccttgctttg
tgacttccaagaacgagtgtctctggaccgacatgctctccaatttcggt
taccctggctaccagtccaaacactacgcctgcatccggcagaagggcgg
ctactgcagctggtaccgaggatgggcccccccggataaaagcatcatca
atgccacagacccc
where x can be t, c, a or g,
or
gcxtgcggxtgctcgcccagccacccccaggacgccttctgcaactccga
catcgtgatccgggccaaggtggtggggaagaagctggtaaaggaggggc
ccttcggcacgctggtctacaccatcaagcagatgaagatgtaccgaggc
ttcaccaagatgccccatgtgcagtacatccacacggaagcttccgagag
tctctgtggccttaagctggaggtcaacaagtaccagtacctgctgacag
gtcgcgtctatgatggcaagatgtacacggggctgtgcaacttcgtggag
aggtgggaccagctcaccctctcccagcgcaaggggctgaactatcggta
tcacctgggttgtaactgcaagatcaagtcctgctactacctgccttgct
ttgtgacttccaagaacgagtgtctctggaccgacatgctctccaatttc
ggttaccctggctaccagtccaaacactacgcctgcatccggcagaaggg
cggctactgcagctggtaccgaggatgggcccccccggataaaagcatca
tcaatgccacagacccc
where x can be t, c, a or g
or
gcxtgcacatgctcgcccagccacccccaggacgccttctgcaactccga
catcgtgatccgggccaaggtggtggggaagaagctggtaaaggaggggc
ccttcggcacgctggtctacaccatcaagcagatgaagatgtaccgaggc
ttcaccaagatgccccatgtgcagtacatccacacggaagcttccgagag
tctctgtggccttaagctggaggtcaacaagtaccagtacctgctgacag
gtcgcgtctatgatggcaagatgtacacggggctgtgcaacttcgtggag
aggtgggaccagctcaccctctcccagcgcaaggggctgaactatcggta
tcacctgggttgtaac
where x can be t, c, a or g
or
gcxgcxtgcacatgctcgcccagccacccccaggacgccttctgcaactc
cgacatcgtgatccgggccaaggtggtggggaagaagctggtaaaggagg
ggcccttcggcacgctggtctacaccatcaagcagatgaagatgtaccga
ggcttcaccaagatgccccatgtgcagtacatccacacggaagcttccga
gagtctctgtggccttaagctggaggtcaacaagtaccagtacctgctga
caggtcgcgtctatgatggcaagatgtacacggggctgtgcaacttcgtg
gagaggtgggaccagctcaccctctcccagcgcaaggggctgaactatcg
gtatcacctgggttgtaac
where x can be t, c, a or g,
or
tgcggxtgctcgcccagccacccccaggacgccttctgcaactccgacat
cgtgatccgggccaaggtggtggggaagaagctggtaaaggaggggccct
tcggcacgctggtctacaccatcaagcagatgaagatgtaccgaggcttc
accaagatgccccatgtgcagtacatccacacggaagcttccgagagtct
ctgtggccttaagctggaggtcaacaagtaccagtacctgctgacaggtc
gcgtctatgatggcaagatgtacacggggctgtgcaacttcgtggagagg
tgggaccagctcaccctctcccagcgcaaggggctgaactatcggtatca
cctgggttgtaac
where x can be t, c, a or g,
or
gcxtgcggxtgctcgcccagccacccccaggacgccttctgcaactccga
catcgtgatccgggccaaggtggtggggaagaagctggtaaaggaggggc
ccttcggcacgctggtctacaccatcaagcagatgaagatgtaccgaggc
ttcaccaagatgccccatgtgcagtacatccacacggaagcttccgagag
tctctgtggccttaagctggaggtcaacaagtaccagtacctgctgacag
gtcgcgtctatgatggcaagatgtacacggggctgtgcaacttcgtggag
aggtgggaccagctcaccctctcccagcgcaaggggctgaactatcggta
tcacctgggttgtaac
where x can be t, c, a or g.

20. A polynucleotide as in claim 17 comprising a polynucleotide sequence selected from the group consisting of
gcxtgcacatgctcgcccagccacccccaggacgccttctgcaactccga
catcgtgatccgggccaaggtggtggggaagaagctggtaaaggaggggc
ccttcggcacgctggtctacaccatcaagcagatgaagatgtaccgaggc
ttcaccaagatgccccatgtgcagtacatccacacggaagcttccgagag
tctctgtggccttaagctggaggtcaacaagtaccagtacctgctgacag
gtcgcgtctatgatggcaagatgtacacggggctgtgcaacttcgtggag
aggtgggaccagctcaccctctcccagcgcaaggggctgaactatcggta
tcacctgggttgtaactgcaagatcaagtcctgctactacctgccttgct
ttgtgacttccaagaacgagtgtctctggaccgacatgctctccaatttc
ggttaccctggctaccagtccaaacactacgcctgcatccggcagaaggg
cggctactgcagctggtaccgaggatgggcccccccggataaaagcatca
tcaatgccacagacccc
where x can be t, c, a or g,
or
gcxgcxtgcacatgctcgcccagccacccccaggacgccttctgcaactc
cgacatcgtgatccgggccaaggtggtggggaagaagctggtaaaggagg
ggcccttcggcacgctggtctacaccatcaagcagatgaagatgtaccga
ggcttcaccaagatgccccatgtgcagtacatccacacggaagcttccga
gagtctctgtggccttaagctggaggtcaacaagtaccagtacctgctga
caggtcgcgtctatgatggcaagatgtacacggggctgtgcaacttcgtg
gagaggtgggaccagctcaccctctcccagcgcaaggggctgaactatcg
gtatcacctgggttgtaactgcaagatcaagtcctgctactacctgcctt
gctttgtgacttccaagaacgagtgtctctggaccgacatgctctccaat
ttcggttaccctggctaccagtccaaacactacgcctgcatccggcagaa
gggcggctactgcagctggtaccgaggatgggcccccccggataaaagca
tcatcaatgccacagacccc
where x can be t, c, a or g,
or
tgcggxtgctcgcccagccacccccaggacgccttctgcaactccgacat
cgtgatccgggccaaggtggtggggaagaagctggtaaaggaggggccct
tcggcacgctggtctacaccatcaagcagatgaagatgtaccgaggcttc
accaagatgccccatgtgcagtacatccacacggaagcttccgagagtct
ctgtggccttaagctggaggtcaacaagtaccagtacctgctgacaggtc
gcgtctatgatggcaagatgtacacggggctgtgcaacttcgtggagagg
tgggaccagctcaccctctcccagcgcaaggggctgaactatcggtatca
cctgggttgtaactgcaagatcaagtcctgctactacctgccttgctttg
tgacttccaagaacgagtgtctctggaccgacatgctctccaatttcggt
taccctggctaccagtccaaacactacgcctgcatccggcagaagggcgg
ctactgcagctggtaccgaggatgggcccccccggataaaagcatcatca
atgccacagacccc
where x can be t, c, a or g,
or
gcxtgcggxtgctcgcccagccacccccaggacgccttctgcaactccga
catcgtgatccgggccaaggtggtggggaagaagctggtaaaggaggggc
ccttcggcacgctggtctacaccatcaagcagatgaagatgtaccgaggc
ttcaccaagatgccccatgtgcagtacatccacacggaagcttccgagag
tctctgtggccttaagctggaggtcaacaagtaccagtacctgctgacag
gtcgcgtctatgatggcaagatgtacacggggctgtgcaacttcgtggag
aggtgggaccagctcaccctctcccagcgcaaggggctgaactatcggta
tcacctgggttgtaactgcaagatcaagtcctgctactacctgccttgct
ttgtgacttccaagaacgagtgtctctggaccgacatgctctccaatttc
ggttaccctggctaccagtccaaacactacgcctgcatccggcagaaggg
cggctactgcagctggtaccgaggatgggcccccccggataaaagcatca
tcaatgccacagacccc
where x can be t, c, a or g
or
gcxtgcacatgctcgcccagccacccccaggacgccttctgcaactccg
acatcgtgatccgggccaaggtggtggggaagaagctggtaaaggaggg
gcccttcggcacgctggtctacaccatcaagcagatgaagatgtaccga
ggcttcaccaagatgccccatgtgcagtacatccacacggaagcttccg
agagtctctgtggccttaagctggaggtcaacaagtaccagtacctgct
gacaggtcgcgtctatgatggcaagatgtacacggggctgtgcaacttc
gtggagaggtgggaccagctcaccctctcccagcgcaaggggctgaact
atcggtatcacctgggttgtaac
where x can be t, c, a or g
or
gcxgcxtgcacatgctcgcccagccacccccaggacgccttctgcaact
ccgacatcgtgatccgggccaaggtggtggggaagaagctggtaaagga
ggggcccttcggcacgctggtctacaccatcaagcagatgaagatgtac
cgaggcttcaccaagatgccccatgtgcagtacatccacacggaagctt
ccgagagtctctgtggccttaagctggaggtcaacaagtaccagtacct
gctgacaggtcgcgtctatgatggcaagatgtacacggggctgtgcaac
ttcgtggagaggtgggaccagctcaccctctcccagcgcaaggggctga
actatcggtatcacctgggttgtaac
where x can be t, c, a or g,
or
tgcggxtgctcgcccagccacccccaggacgccttctgcaactccgaca
tcgtgatccgggccaaggtggtggggaagaagctggtaaaggaggggcc
cttcggcacgctggtctacaccatcaagcagatgaagatgtaccgaggc
ttcaccaagatgccccatgtgcagtacatccacacggaagcttccgagag
tctctgtggccttaagctggaggtcaacaagtaccagtacctgctgaca
ggtcgcgtctatgatggcaagatgtacacggggctgtgcaacttcgtgg
agaggtgggaccagctcaccctctcccagcgcaaggggctgaactatcg
gtatcacctgggttgtaac
where x can be t, c, a or g,
or
gcxtgcggxtgctcgcccagccacccccaggacgccttctgcaactccg
acatcgtgatccgggccaaggtggtggggaagaagctggtaaaggagggg
cccttcggcacgctggtctacaccatcaagcagatgaagatgtaccgagg
cttcaccaagatgccccatgtgcagtacatccacacggaagcttccgaga
gtctctgtggccttaagctggaggtcaacaagtaccagtacctgctgaca
ggtcgcgtctatgatggcaagatgtacacggggctgtgcaacttcgtgga
gaggtgggaccagctcaccctctcccagcgcaaggggctgaactatcggt
atcacctgggttgtaac
where x can be t, c, a or g.

21. A recombinant polynucleotide suitable for expressing a mutated TIMP-3 polypeptide according to claim 1.

22. A host cell comprising a polynucleotide selected from the group consisting of a polynucleotide encoding a mutant TIMP-3 polypeptide according to claim 1 and a recombinant polynucleotide suitable for expressing a mutated TIMP-3 polypeptide according to claim 1.

23. A method of making a mutated TIMP-3 polypeptide according to claim 1, the method comprising:
(a) culturing a host cell comprising a polynucleotide selected from the group consisting of a polynucleotide encoding a mutant TIMP-3 polypeptide according to claim 1 and a recombinant polynucleotide suitable for expressing a mutated TIMP-3 polypeptide according to claim 1 which expresses said mutated TIMP-3 polypeptide; and
(b) isolating said mutated TIMP-3 polypeptide.

24. A mutated TIMP-3 polypeptide obtainable by the method of claim 23.

25. A method of identifying a compound that is expected to inhibit an ADAM metalloproteinase (for example TACE, ADAMTS-4 or ADAMTS-5) to a greater extent than an MMP (matrix metalloproteinase), comprising:
(a) comparing a structure of a test compound with a structure of at least the N-terminal 4, 5, 6, 7, 8, 9 or 10 amino acids of a mutant TIMP-3 polypeptide according to claim 1; and
(b) selecting a compound that is considered to have a structure similar to that of the at least the N-terminal 4, 5, 6, 7, 8, 9 or 10 amino acids of the said mutant TIMP-3 polypeptide.

26. A medicament for treating a patient in need of inhibition of one or more ADAMs, for example TACE (TNFα Converting Enzyme), ADAMTS4 or ADAMTS5, said medicament comprising a material selected from the group consisting of a polypeptide according to claim 1 and a polynucleotide encoding a mutant TIMP-3 polypeptide according to claim 1 and a recombinant polynucleotide suitable for expressing a mutated TIMP-3 polypeptide according to claim 1.

27. A medicament as in claim 26 wherein the medicament is specified for treating rheumatoid arthritis, osteoarthritis, osteopenia, osteolysis, osteoporosis, Crohn's disease, ulcerative colitis, degenerative cartilage loss, sepsis, AIDS, HIV infection, graft rejection, anorexia, inflammation, congestive heart failure, post-ischaemic reperfusion injury, inflammatory disease of the central nervous system, inflammatory bowel disease, insulin resistance, septic shock, haemodynamic shock, sepsis syndrome, malaria, mycobacterial infection, meningitis, psoriasis, fibrotic diseases, cachexia, graft rejection, cancer, diseases involving angiogenesis, autoimmune diseases, skin inflammatory diseases, multiple sclerosis, radiation damage, hyperoxic alveolar injury, periodontal disease, non-insulin dependent diabetes mellitus, neovascularization, rubeosis iridis, neovascular glaucoma, age-related macular degeneration, diabetic retinopathy, ischemic retinopathy, or retinopathy of prematurity.

28. A method of treating a patient in need of inhibition of one or more ADAMs, for example TACE (TNFα Converting Enzyme), ADAMTS-4 or ADAMTS-5, comprising administering to the patient a therapeutically effective amount of a medicament according to claim 26.


SEQUENCE LISTING (29 sequences)

SEQ ID NO: 1 - DNA, 31 bp, Artificial Sequence
  T2G Forward Primer
aaaacatatgtgcggatgctcgcccagccac

SEQ ID NO: 2 - DNA, 34 bp, Artificial Sequence
  1Ala Forward Primer
aaaacatatggcatgcacatgctcgcccagccac

SEQ ID NO: 3 - DNA, 30 bp, Artificial Sequence
  Reverse Primer for N-TIMP-3 mutants
aaaagcggccgcgttacaacccaggtgata

SEQ ID NO: 4 - protein, 189 aa, Homo sapiens
  MUTAGEN (1) ... (1): Xaa can be any one of Ala, Asp, Glu, Phe, Gly, His, Ile, Lys, Leu, Met, Asn, Pro, Gln, Arg, Ser, Thr, Val, Trp or Tyr
XCTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 5 - protein, 190 aa, Homo sapiens
AACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 6 - protein, 188 aa, Homo sapiens
  MUTAGEN (2) ... (2): Xaa can be any one of Gly, Ala, Asp, Glu, Phe, His, Ile, Lys, Asn, Pro, Gln, Arg, Val or Trp
CXCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 7 - protein, 189 aa, Homo sapiens
  MUTAGEN (1) ... (1): Xaa can be any one of Ala, Asp, Glu, Phe, Gly, His, Ile, Lys, Leu, Met, Asn, Pro, Gln, Arg, Ser, Thr, Val, Trp or Tyr
XCXCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 8 - protein, 122 aa, Homo sapiens
  MUTAGEN (1) ... (1): Xaa can be any one of Ala, Asp, Glu, Phe, Gly, His, Ile, Lys, Leu, Met, Asn, Pro, Gln, Arg, Ser, Thr, Val, Trp or Tyr
XCTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCN

SEQ ID NO: 9 - protein, 123 aa, Homo sapiens
AACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCN

SEQ ID NO: 10 - protein, 121 aa, Homo sapiens
  MUTAGEN (2) ... (2): Xaa can be any one of Gly, Ala, Asp, Glu, Phe, His, Ile, Lys, Asn, Pro, Gln, Arg, Val or Trp
CXCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCN

SEQ ID NO: 11 - protein, 122 aa, Homo sapiens
  MUTAGEN (1) ... (1): Xaa can also be any one of Asp, Glu, Phe, Gly, His, Ile, Lys, Leu, Met, Asn, Pro, Gln, Arg, Ser, Thr, Val, Trp or Tyr
XCXCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCN

SEQ ID NO: 12 - DNA, 567 bp, Homo sapiens
  misc_feature (3) ... (3): Can be any one of T, C, A or G
gcntgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccc

SEQ ID NO: 13 - DNA, 570 bp, Homo sapiens
  misc_feature (3) ... (3): Can be any one of T, C, G or A
gcngcntgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccc

SEQ ID NO: 14 - DNA, 564 bp, Homo sapiens
  misc_feature (6) ... (6): Can be any one of T, C, A or G
tgcggntgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccc

SEQ ID NO: 15 - DNA, 567 bp, Homo sapiens
  misc_feature (3) ... (3): Can be any one of T, C, A or G
gcntgcggntgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccc

SEQ ID NO: 16 - DNA, 366 bp, Homo sapiens
  misc_feature (3) ... (3): Can be any one of T, C, A or G
gcntgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaac

SEQ ID NO: 17 - DNA, 369 bp, Homo sapiens
  misc_feature (3) ... (3): Can be any one of T, C, A or G
gcngcntgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaac

SEQ ID NO: 18 - DNA, 363 bp, Homo sapiens
  misc_feature (6) ... (6): Can be any one of T, C, A or G
tgcggntgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaac

SEQ ID NO: 19 - DNA, 366 bp, Homo sapiens
  misc_feature (3) ... (3): Can be any one of T, C, A or G
gcntgcggntgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaac

SEQ ID NO: 20 - DNA, 636 bp, Homo sapiens
atgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctga

SEQ ID NO: 21 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 22 - DNA, 573 bp, Homo sapiens
  misc_feature (6) ... (6): Can be any one of T, C, A or G
atggcntgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctga

SEQ ID NO: 23 - DNA, 570 bp, Homo sapiens
  misc_feature (9) ... (9): Can be any one of T, C, A or G
atgtgcggntgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctga

SEQ ID NO: 24 - DNA, 573 bp, Homo sapiens
  misc_feature (6) ... (6): Can be any one of T, C, A or G
atggcntgcggntgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctga

SEQ ID NO: 25 - DNA, 576 bp, Homo sapiens
  misc_feature (6) ... (6): Can be any one of T, C, A or G
atggcngcntgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctga

SEQ ID NO: 26 - DNA, 372 bp, Homo sapiens
  misc_feature (6) ... (6): Can be any one of T, C, A or G
atggcntgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactga

SEQ ID NO: 27 - DNA, 369 bp, Homo sapiens
  misc_feature (9) ... (9): Can be any one of T, C, A or G
atgtgcggntgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactga

SEQ ID NO: 28 - DNA, 372 bp, Homo sapiens
  misc_feature (6) ... (6): Can be any one of T, C, A or G
atggcntgcggntgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactga

SEQ ID NO: 29 - DNA, 375 bp, Homo sapiens
  misc_feature (6) ... (6): Can be any one of T, C, A or G
atggcngcntgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactga